(19) United States
(12) Patent Application Publication
(10) Pub. No.: US 2025/0188244 A1
(43) Pub. Date: Jun. 12, 2025

(54) Methods of Purification of Recycled Monomers, and Recycled Monomers and Uses Thereof

(71) Applicant: Cornell University, Ithaca, NY (US)

(72) Inventors: Phillip J. Milner, Ithaca, NY (US); Abigail K. Nason, Ithaca, NY (US); Ronald T. Jerozal, Ithaca, NY (US); Jin Suntivich, Ithaca, NY (US)

(21) Appl. No.: 19/040,219

(22) Filed: Jan. 29, 2025

Related U.S. Application Data
(63) Continuation-in-part of application No. PCT/US2023/071352, filed on Jul. 31, 2023.
(60) Provisional application No. 63/567,396, filed on Mar. 19, 2024.
(60) Provisional application No. 63/393,431, filed on Jul. 29, 2022.

(51) Int. Cl. C08J 11/14; C07C 51/43

(52) U.S. Cl. CPC C08J 11/14; C07C 51/43; C08J 2367/02

(57) ABSTRACT

Methods of purifying impure monomers or forming purified monomers. In various examples, a method recycles one or more polymer(s). In various examples, a method comprises forming metal complex(es), metal organic framework(s) (MOF(s)), coordination polymer network(s), or the like, or any combination thereof using impure monomer(s). In various examples, the metal complex(es), (MOF(s), coordination polymer network(s), or the like, or any combination thereof are decomposed to provide purified monomer(s). In various examples, the purified monomer(s) is/are used in a subsequent polymerization reaction or the like. In various examples, a polymer is formed from one or more purified monomer(s). In various examples, an article of manufacture comprises one or more of the polymer(s). In various examples, the article is a container, a structural element, or a surface element, or the like.

### CROSS-REFERENCE TO RELATED APPLICATIONS

This application claims the benefit of U.S. Provisional Patent Application No. 63/567,396, filed Mar. 19, 2024 and entitled “Methods of Purification of Recycled Monomers, and Recycled Monomers and Uses Thereof,” and is a continuation-in-part of International Patent Application No. PCT/US2023/071352, filed Jul. 31, 2023 and entitled “Methods of Purification of Recycled Monomers, and Recycled Monomers and Uses Thereof,” which claims the benefit of U.S. Provisional Patent Application No. 63/393,431, filed Jul. 29, 2022 and entitled “Methods of Purification of Recycled Monomers, and Recycled Monomers and Uses Thereof.” The entire contents of the above-identified priority applications are hereby fully incorporated herein by reference in their entirety.

### STATEMENT REGARDING FEDERALLY SPONSORED RESEARCH

This invention was made with government support under grant number 2139899 awarded by the National Science Foundation. The government has certain rights in the invention.

### BACKGROUND OF THE DISCLOSURE

Polyethylene terephthalate (PET) textiles are among the largest categories of in-production plastics. PET is a common polymer in textiles, packaging, and bottles. Textile global demand is rapidly increasing, with over 60 Mt produced in 2018 alone. Therefore, the extraction of PET in post-consumer textiles plays an important role in the sustainability of PET manufacturing.

Because they are blended with other components, such as cotton, dyes, and additives, depolymerizing these PET blends leave impurities trapped in the extracted monomers (terephthalic acid, “TPA”). PET is a major contributor to global plastic waste. Recycling has the potential to alleviate this concern. However, existing approaches (mechanical recycling) need clean starting materials.

Chemical recycling can give a second life to these impurity-laden plastics. While clean PET bottles can be mechanically recycled through a series of cleaning steps or melting and extrusion, blended PET products such as textiles, which contain dyes and other additives, are not yet suitable for mechanical recycling approaches. Mechanical recycling can recycle pure PET waste into new PET materials through melting and extrusion; however, challenges arise when PET is blended with additives such as dyes and pigments. In such cases, mechanical recycling is unsuitable. Because these impurities can create undesired attributes in the final product, reusing PET from post-consumer textiles requires impurity separation.

Chemical recycling, which uses reagents to depolymerize PET into monomers, can depolymerize blended plastics; however, separating the impurities post-depolymerization is not straightforward. Post-depolymerization purification to remove impurities is necessary essential because a small percentage of remaining additives can cause undesired properties in the final product. This recognition has driven efforts to separate impurities from monomers from depolymerized plastics; however, these steps bring extra processes and complications, incurring additional costs. Considering that the textile industry produces over 60% of global PET supply, devising a strategy to separate impurities from recycled monomers is a key step toward mitigating their environmental impact, especially given the growing trend of fast fashion.

Polyester and cotton (polycotton) blended fabrics are among the most common textiles being produced. Alkaline hydrolysis can chemically recycle PET fibers in polycotton to form terephthalic acid (TPA) and ethylene glycol (EG) monomers while leaving cotton intact. Alkaline hydrolysis can depolymerize colored PET bottles; however, the impurities present in the starting material stayed attached to the recycled TPA (rTPA) even after the purification step. These impurities are a significant concern given that they can affect the quality, appearance, and safety of the recycled products.

The methods for purifying TPA are energy and reagent intensive. Improvement in the purity of the monomers can be made through recrystallization, for example, by using organic solvents and chemical treatments. The most common approach is to take advantage of the temperature-dependent solubility of TPA. With limited solubility in water, the water-based method requires a controlled cooling step from an autoclave for impurity rejection. Alternatively, one can take advantage of the increased solubility of TPA in organic solvents such as N,N-dimethylacetamide. The drawback of this approach is the need for a solvent recovery system. Pressurized water heating can take advantage of the increased BDC solubility; however, recrystallization without re-trapping impurities or damaging the BDC is challenging. Another possibility is to add excess methanol in the depolymerization process (‘methanolysis’) to form dimethyl terephthalate (DMT) that has a lower melting temperature than TPA. This approach allows DMT purification via crystallization; however, it requires additional methanol recovery and purification steps.

### SUMMARY OF THE DISCLOSURE

The present disclosure provides, inter alia, methods of forming one or more purified monomer(s) and polymers formed from at least a purified monomer or purified monomers formed by a method of the present disclosure. The present disclosure also provides articles of manufacture comprising one or more of the polymers. Non-limiting examples of the methods, the purified monomers, the polymers, and the articles of manufacture are provided herein.

In various examples, the present disclosure provides a method of forming one or more purified monomer(s) and/or structural analog(s) comprises forming one or more metal complex(es), one or more metal organic framework(s) (MOF(s)), one or more coordination polymer network(s), or any combination thereof using one or more impure monomer(s), structural analog(s) thereof, or any combination thereof; optionally, isolating the metal complex(es), one or more metal organic framework(s) (MOF(s)), coordination polymer network(s); optionally, decomposing the metal complex(es), metal organic framework(s), coordination polymer network(s), where purified monomer(s) and/or structural analog(s) thereof are formed; and optionally, isolating the purified monomer(s) and/or structural analog(s). In various examples, the method further comprising providing the impure monomer(s) and/or structural analog(s) thereof. In various examples, the polymer(s) is/are chosen from organic polymers, biopolymers, and the like, and any combination thereof. In various examples, the impure monomer(s) and/or structural analog(s) thereof is/are organic acid(s), polyol(s), amine(s), or the like, or any combination thereof. In various examples, the method is a one-pot method or comprises use of a single reaction mixture. In various examples, the impure monomer(s) and/or structural analog(s) thereof is/are formed from a textile, an article of manufacture, or the like, or any combination thereof. In various examples, the forming the metal complex(es), metal organic framework(s), coordination polymer network(s), or the like, or any combination thereof comprises contacting the impure monomer(s) and/or structural analog(s) thereof with one or more metal precursor(s), where the metal complex(es), metal organic framework(s), coordination polymer network(s), or the like are formed. In various examples, the decomposing the metal complex(es), metal organic framework(s), coordination polymer network(s), or the like, or any combination thereof comprises contacting the metal complex(es), metal organic framework(s), coordination polymer network(s), or the like, or any combination thereof with one or more acid(s), one or more base(s), one or more organic solvent(s), or one or more ionic liquid(s), or any combination thereof. In various examples, the decomposing the metal complex(es), metal organic framework(s), coordination polymer network(s), or the like, or any combination thereof comprises contacting the metal complex(es), metal organic framework(s), coordination polymer network(s), or the like, or any combination thereof with one or more acid(s), where metal precursor(s) or structural analog(s) thereof are formed. In various examples, the impure monomer(s) and/or structural analog(s) thereof comprise one or more impurit(ies) and the purified monomer(s) and/or structural analog(s) is/are substantially free or free of the impurit(ies) and/or the impure monomer(s) and/or structural analog(s) thereof comprise one or more additive(s) and the purified monomer(s) and/or structural analog(s) is/are substantially free or free of the additive(s). In various examples, the impure monomer(s) and/or structural analog(s) are less than 99% pure by weight. In various examples, the impure monomer(s) and/or structural analog(s) are less than 90% pure by weight (e.g., based on the total weight of the impure monomer(s), impurit(ies), if present, and additive(s), if present. In various examples, the purified monomer(s) is/are substantially colorless or colorless. In various examples, the method further comprises polymerizing the purified monomer(s) or structural analog(s) thereof.

In various examples, the present disclosure provides a polymer formed from at least a purified monomer or purified monomers and/or a structural analog or analogs thereof formed by a method of the present disclosure. In various examples, the polymer is a homopolymer or a copolymer. In various examples, the polymer is chosen from organic polymers and biopolymers, and the like. In various examples, the polymer is a polyester (such as, for example, a poly(terephthalate), a poly(propylene terephthalate), a poly(butylene terephthalate), a polylactic acid, or the like), a polyamide (such as, for example, a nylon or the like), a polyalcohol, a polyurethane, a polyurea, a polycarbonate, a polyether ether ketone, a polyether ether ketone ketone, a polyether ketone ketone, a polyetherimide, a polystyrene, or the like, or any combination thereof. In various examples, the polymer is in the form of a textile, an article of manufacture (such as, for example, a container (such as, for example, a bottle, a food tray, a food packaging, or the like), a rug/carpet(s), a structural element, a surface element (such as, for example, a component or part from an automobile, a plane, or the like), or the like. In various examples, the polymer is substantially free of impurit(ies) and/or additive(s) present in the impure monomer(s) and/or structural analog(s) from which the purified monomer(s) and/or structural analog(s) thereof was/were formed.

In various examples, the present disclosure provides an article of manufacture comprising one or more polymer(s) of the present disclosure (e.g., formed from at least a purified monomer or purified monomers and/or a structural analog or analogs thereof formed by a method of the present disclosure). In various examples, the article of manufacture is a container, a structural element, or a surface element, or the like. In various examples, the container is a bottle, a food tray, or a food packaging, or the like. In various examples, the surface element is a component or part from an automobile, or a plane, or the like.

### BRIEF DESCRIPTION OF THE FIGURES

For a fuller understanding of the nature and objects of the disclosure, reference should be made to the following detailed description taken in conjunction with the accompanying figures.

FIG. 1 shows a schematic of the purification cycle of terephthalic acid recovered from waste textiles.

FIG. 2 shows (a) Powder X-ray diffraction of recycled TPA from red polycotton (“rTPA”), MIL-53(Ga) MOF synthesized from rTPA (“MOF”), and purified TPA from MOF disassembly (“pTPA”), (b) photo, and (c) UV-Vis spectra of rTPA and pTPA in DMSO.

FIG. 3 shows (a) photo and (b) UV-Vis spectra of rTPA obtained from blue polycotton and the corresponding pTPA in DMSO. (c) photo and (d) UV-Vis spectra of rTPA obtained from green polycotton and the corresponding pTPA dissolved in DMSO.

FIG. 4 shows PXRD patterns (wavelength (λ)=1.5406 Å) of MIL-53(Ga) synthesized from rTPA (showing here rTPA from red fabric) and reference MIL-53(Ga) pattern (Vougo-Zanda et al).

FIG. 5 shows TGA curves of recycled TPA (rTPA), purified TPA (pTPA), and commercial TPA (cTPA) for TPA from (a) red fabric, (b) blue fabric, and (c) green fabric. The thermogravimetric decomposition profiles were collected on a TA Instruments TGA Q5000 under an atmosphere of N₂ and with a ramp rate of 3° C./min.

FIG. 6 shows FTIR infrared spectra of recycled TPA (rTPA), purified TPA (pTPA), and commercial TPA (cTPA) for TPA from (a) red fabric, (b) blue fabric, and (c) green fabric. The spectra were collected on a Bruker Tensor II IR spectrometer equipped with a diamond Attenuated Total Reflectance (ATR) attachment.

FIG. 7 shows (a) ¹H NMR and (b) ¹³C NMR (500 MHz, DMSO-d₆) of recycled TPA (rTPA), purified TPA (pTPA), and commercial TPA (cTPA) for TPA from red fabric. Peaks are color-coded based on corresponding protons and carbons in the TPA molecule.

FIG. 8 shows SEM images (Zeiss Gemini 500 Scanning Electron Microscope) of MIL-53(Ga) crystals synthesized from (a) rTPA from red fabric, (b) rTPA from blue fabric, and (c) rTPA from green fabric. Samples are carbon-sputtered.

FIG. 9 shows TPA purification with no organic solvents. (a) Powder X-ray diffraction patterns (λ=1.5406 Å) of purified TPA from MOF disassembly (“pTPA”) and MIL-53(Ga) MOF synthesized from rTPA (“MIL-53 (Ga)”), (b) photo of rTPA versus pTPA in DMSO, and (c) UV-Vis spectra of commercial TPA and pTPA.

FIG. 10 shows PXRD pattern (λ=1.5406 Å) of MIL-53(Ga) made from recycled Ga(NO₃)₃ and rTPA from red polycotton fabric. The recycled Ga(NO₃)₃ was obtained by evaporating the excess liquid from the effluent of a MIL-53(Ga) disassembly from an earlier experiment of reactive crystallization.

FIG. 11 shows X-ray photoelectron spectroscopy (XPS) of pTPA from red polycotton fabric showing less than 0.1 at % Ga impurities. Samples were analyzed using a Scienta Omicron ESCA-2SR Spectrometer with operating pressure ca. 1×10⁻⁹ Torr. Monochromatic Al Kα x rays (1486.6 eV) with photoelectrons collected from a 1.1 mm diameter analysis spot. Photoelectrons were collected at a 900 emission angle with source to analyzer angle of 54.7°. A hemispherical analyzer determined electron kinetic energy, using a pass energy of 200 eV for wide/survey scans, and 50 eV for high resolution scans. A flood gun was used for charge neutralization of non-conductive samples.

FIG. 12 shows an example of a system of the present disclosure.

FIG. 13 shows (a) a schematic of a procedure for recovering purified benzene-1,4-dicarboxylic acid (pBDC) from waste PET textiles. The PET textile is a black post-consumer fleece jacket made from polyethylene terephthalate (PET) plastic fibers and (b) a schematic of a one-step procedure for recovering purified benzene-1,4-dicarboxylic acid (pBDC) from waste PET textiles. The PET textile is a black post-consumer fleece jacket made from polyethylene terephthalate (PET) plastic fibers.

FIG. 14 shows powder X-ray diffraction of the PET fabric precursor (top) and Zn(BDC)(H₂O) MOF synthesized from red PET fabric under the conditions of 2:1 Zn:BDC, 210° C., and 12 h (bottom). The simulated PXRD pattern of each known phase based on single-crystal XRD analysis is included for reference.

FIG. 15 shows (a) a kinetic phase diagram showing the effect of Zn:BDC molar ratio and reaction time on the synthesized Zn-MOF structure. The grey rings represent unreacted BDC as detected by PXRD. Black dashed lines represent phase transitions. Gray dashed line represents predicted phase transition. (b) Time-course PXRD changes of 1:1 Zn:BDC molar ratio MOF syntheses with increasing reaction time. (c) Time-course PXRD changes of 2:1 Zn:BDC molar ratio MOF syntheses with increasing reaction time. Zn(BDC)(H₂O), Zn(BDC)(H₂O)₂, and Zn₂(OH)₂(BDC)(H₂O) structures have been confirmed by PXRD. Zn(BDC) structure was unable to be resolved by Pawley Refinements. This proposed structure is based on TGA analysis and ATR-IR.

FIG. 16 shows powder X-ray diffractions showing the products formed at different reaction times when using (a) PET fabric and (b) commercial BDC (cBDC) as the organic linker source and Zn(NO₃)₂·6H₂O as the metal salt in a 2:1 molar ratio at 210° C. The PXRDs in (a) at 6 h, 12 h, and 18 h showing the structure Zn(BDC)(H₂O) have corresponding Pawley refinements. The 4 h, 2:1 Zn:BDC PXRD shows BDC and unreacted PET fabric.

FIG. 17 shows (a) attenuated total reflectance infrared (ATR-IR) spectra of commercial BDC (cBDC), purified BDC (pBDC) made from the disassembly of 1:1 Zn:BDC 12 h Zn(BDC)(H₂O) MOF, and hydrothermal BDC (hBDC) made from the hydrothermal hydrolysis of black fleece post-consumer PET fabric. (b) Powder X-ray diffraction of pBDC and hBDC. (c) Photos and (d) UV-vis spectra of hBDC and pBDC in DMSO.

### DETAILED DESCRIPTION OF THE DISCLOSURE

Although claimed subject matter will be described in terms of certain examples and embodiments, other examples and embodiments, including examples that do not provide all of the benefits and features set forth herein, are also within the scope of this disclosure. Various structural, logical, and process step changes may be made without departing from the scope of the disclosure.

As used herein, unless otherwise indicated, “about”, “substantially”, or “the like”, when used in connection with a measurable variable (such as, for example, a parameter, an amount, a temporal duration, or the like) or a list of alternatives, is meant to encompass variations of and from the specified value including, but not limited to, those within experimental error (which can be determined by, e.g., a given data set, an art accepted standard, etc. and/or with, e.g., a given confidence interval (e.g. 90%, 95%, or more confidence interval from the mean), such as, for example, variations of +/−10% or less, +1-5% or less, +/−1% or less, and +/−0.1% or less of and from the specified value), insofar such variations in a variable and/or variations in the alternatives are appropriate to perform in the instant disclosure. As used herein, the term “about” may mean that the amount or value in question is the exact value or a value that provides equivalent results or effects as recited in the claims or taught herein. That is, it is understood that amounts, sizes, compositions, parameters, and other quantities and characteristics are not and need not be exact, but may be approximate and/or larger or smaller, as desired, reflecting tolerances, conversion factors, rounding off, measurement error, or the like, or other factors known to those of skill in the art such that equivalent results or effects are obtained. In general, an amount, size, composition, parameter, or other quantity or characteristic, or alternative is “about” or “the like,” whether or not expressly stated to be such. It is understood that where “about,” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or subranges encompassed within that range as if each numerical value and subrange is explicitly recited. To illustrate, a numerical range of “0.1% to 5%” should be interpreted to include not only the explicitly recited values of 0.1% to 5%, but also, unless otherwise stated, include individual values (e.g., 1%, 2%, 3%, and 4%) and the subranges (e.g., 0.5% to 1.1%; 0.5% to 2.4%; 0.5% to 3.2%, and 0.5% to 4.4%, and other possible subranges) within the indicated range. It is also understood (as presented above) that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about, it will be understood that the particular value forms a further disclosure. For example, if the value “about 10” is disclosed, then “10” is also disclosed.

As used herein, unless otherwise stated, the term “group” refers to a chemical entity that is monovalent (i.e., has one terminus that can be (is) covalently bonded to other chemical species), divalent, or polyvalent (i.e., has two or more termini that can be (are) covalently bonded to other chemical species). The term “group” also includes radicals (e.g., monovalent radicals and multivalent radicals, such as, for example, divalent radicals, trivalent radicals, and the like). Illustrative examples of groups include:

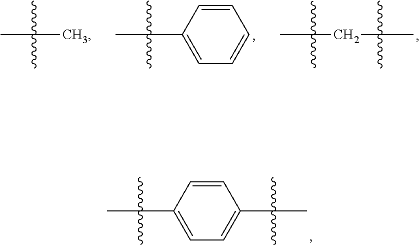

and the like.

As used herein, unless otherwise stated, the term “structural analog” refers to any impure monomer, purified monomer, polymer, metal precursor, solvent, or the like, or any portion thereof (such as, for example, one or more group(s) thereof or the like) if one atom or group of atoms, functional group or functional groups, or substructure or substructures is/are replaced with another atom or group of atoms, functional group or functional groups, substructure or substructures, or the like. In various examples, the term “structural analog” refers to any group that is derived from an original impure monomer, purified monomer, polymer, metal precursor, solvent, or the like, or any portion thereof (such as, for example, one or more group(s) thereof or the like) by a chemical reaction, where the impure monomer, the purified monomer, the polymer, the metal precursor, the solvent, or the like, or the portion thereof (such as, for example, the one or more group(s) thereof or the like) is modified or partially substituted such that at least one structural feature of the original impure monomer, purified monomer, polymer, metal precursor, solvent, or the like, or any portion thereof (such as, for example, one or more group(s) thereof or the like) is retained.

The present disclosure provides, inter alia, methods of obtaining one or more purified monomer(s) or obtaining one or more molecul(es) of interest from one or more polymer(s). The present disclosure also provides uses of purified monomer(s) and molecule(s) of interest obtained using the methods.

In an aspect, the present disclosure provides obtaining one or more purified monomer(s) or structural analog(s) thereof or obtaining one or more molecule(s) of interest or structural analog(s) thereof from one or more polymer(s). In various examples, the methods are polymer recycling methods. In various examples, a method recycles one or more polymer(s) (e.g., purifies recycled monomer(s) or structural analog(s) thereof obtained from polymer(s)). In various examples, a method comprises one or more reactive crystallization(s) or the like. In various examples, a reactive crystallization comprises forming crystals comprising (or of) metal complex(es), metal organic framework(s) (MOF(s)), coordination polymer network(s), or the like, or any combination thereof at or about a purification temperature (e.g., a temperature at which substantially all or all of the impurit(ies) and/or additive(es) of the impure monomer(s) and/or structural analog(s) thereof are not trapped in the crystals during crystallization or during cooling of the crystals). In various examples, a method does not comprise any other monomer purification process(es) or step(s) (such as, for example, monomer pre-purification process(es) or step(s) or the like). In various examples, a purified monomer or monomers and/or a structural analog or analogs thereof (or a composition comprising one or more monomer(s) and/or structural analog(s) thereof) (e.g., recycled monomer(s)) or purified monomer(s)) is/are formed by a method of the present disclosure. Non-limiting examples of the methods are disclosed herein.

A method can recycle various polymers. In various examples, a polymer is a homopolymer, a copolymer, or the like. In various examples, a method is used to recycle a plurality of polymers, where each polymer is structurally distinct from the other polymers. In various examples, polymer(s) is/are chosen from polyester(s) (such as, for example, poly(terephthalate)(s), poly(propylene terephthalate)(s), poly(butylene terephthalate)(s), polylactic acid(s), and the like), polyamide(s) (such as, for example, nylon(s) and the like), polyalcohol(s), polyurethane(s), polyurea(s), polycarbonate(s), polyether ether ketone(s), polyether ether ketone ketone(s), polyether ketone ketone(s), polyetherimide(s), polystyrene(s), and the like, and any combination thereof, and/or the impure monomer(s) (which may be recycled monomer(s) or the like) (or impure molecule(s) of interest), structural analog(s) thereof, or any combination thereof is/are chosen from organic acids (such as, for example, terephthalic acid, isophthalic acid, adipic acid, lactic acid, adipic acid, and the like), polyols (which may be diols or the like) (such as, for example, ethylene glycol, propanediol, butanediol, and the like), organic amines (which may be organic diamines or the like) (such as, for example, methylenedianiline, hexamethylenediamine, and the like), bisphenol A, hydroquinone, structural analogs thereof, and the like and any combination thereof.

In various examples, a method recycles one or more textile(s), one or more article(s) of manufacture (such as, for example, container(s) (such as, for example, bottle(s), food tray(s), food packaging(s), or the like), rug(s)/carpet(s), structural element(s), surface element(s) (such as, for example, component(s) or part(s) from an automobile, a plane, or the like), or the like, or any combination thereof.

In various examples, a method of recycling one or more polymer(s) (e.g., purifying recycled monomer(s) obtained from polymer(s)) (or obtaining one or more molecule(s) of interest, one or more purified monomer(s) (which may be target monomer(s)), or the like, or structural analog(s) thereof, or any combination thereof (e.g., from a polymer/polymers or the like)) comprises: forming one or more metal complex(es), one or more metal organic framework(s) (MOF(s)), one or more coordination polymer network(s), or the like, or any combination thereof comprising one or more impure monomer(s) (which may be recycled monomer(s) or the like) (or impure molecule(s) of interest), structural analog(s) thereof, or any combination thereof; optionally, isolating the metal complex(es), one or more metal organic framework(s) (MOF(s)), coordination polymer network(s), or the like; optionally, decomposing (e.g., a portion, substantially all, or all of) the metal complex(es), metal organic framework(s), coordination polymer network(s), or the like, where purified monomer(s) (e.g., free monomer(s) or the like) (or purified molecule(s) of interest), or structural analog(s) thereof are formed; and optionally, isolating (e.g., a portion, substantially all, or all of) the purified monomer(s) (or purified molecule(s) of interest) or the structural analog(s) thereof.

Forming one or more metal complex(es), one or more metal organic framework(s) (MOF(s)), one or more coordination polymer network(s), or the like, or any combination thereof, optionally, isolating the metal complex(es), the metal organic framework(s) (MOF(s)), coordination polymer network(s), or the like, or the combination thereof; optionally, decomposing (e.g., a portion, substantially all, or all of) the metal complex(es), metal organic framework(s), coordination polymer network(s), or the like, or the combination thereof; and optionally, isolating (e.g., a portion, substantially all, or all of) the purified monomer(s) (or purified molecule(s) of interest) or the structural analog(s) thereof may be repeated a desired number of times. In various examples, the repeated forming(s) is/are carried out using the same metal complex(es), metal organic framework(s) (MOF(s)), coordination polymer network(s), or the like, or the combination thereof or two or more different (e.g., compositionally different, structurally different, or both) metal complex(es), metal organic framework(s) (MOF(s)), coordination polymer network(s), or the like, or the combination thereof. In various examples, the same purified monomer(s) (or purified molecule(s) of interest) and/or structural analog(s) thereof are obtained or two more different purified monomers (or purified molecules of interest) and/or structural analog(s) thereof are obtained.

In various examples, a method of recycling one or more polymer(s) (e.g., purifying recycled monomer(s) obtained from polymer(s)) (or obtaining one or more molecule(s) of interest, one or more purified monomer(s) (which may be target monomer(s)), or the like, or structural analog(s) thereof, or any combination thereof (e.g., from a polymer/polymers or the like)) comprises forming one or more first metal complex(es), one or more first metal organic framework(s) (MOF(s)), one or more first coordination polymer network(s), or any combination thereof using one or more first impure monomer(s), structural analog(s) thereof, or any combination thereof; optionally, isolating the first metal complex(es), the first metal organic framework(s) (MOF(s)), or the first coordination polymer network(s), or the combination thereof; optionally, decomposing the first metal complex(es), the first metal organic framework(s), first coordination polymer network(s), or the combination thereof, where the purified monomer(s) and/or structural analog(s) thereof are formed; and optionally, isolating the purified monomer(s) and/or the structural analog(s) thereof. In various examples, the method further comprises forming one or more second metal complex(es), one or more second metal organic framework(s) (MOF(s)), one or more second coordination polymer network(s), or any combination thereof using one or more second impure monomer(s), structural analog(s) thereof, or any combination thereof; optionally, isolating the second metal complex(es), the second metal organic framework(s) (MOF(s)), or the second coordination polymer network(s), or the combination thereof; optionally, decomposing the second metal complex(es), the second metal organic framework(s), second coordination polymer network(s), or the combination thereof, where the purified monomer(s) and/or structural analog(s) thereof are formed; and optionally, isolating the second purified monomer(s) and/or the structural analog(s) thereof. In various examples, the forming of the one or more second metal complex(es), the one or more second metal organic framework(s) (MOF(s)), the one or more second coordination polymer network(s), or any combination thereof is carried out after the forming of the first metal complex(es), the first metal organic framework(s) (MOF(s)), the first coordination polymer network(s), or any combination thereof.

In various examples, a method recycles two one or more different polymer(s) or two or more different purifying two or more recycled monomer(s) obtained from polymer(s)) (or obtaining one or more molecule(s) of interest, one or more purified monomer(s) (which may be target monomer(s)), or structural analog(s) thereof, or the like or any combination thereof (e.g., from a polymer/polymers or the like)). If, for example, the starting materials contain or a polymer contains multiple molecules of interest or monomers, the forming (e.g., metal-organic framework crystallization or the like) may occur sequentially or in parallel. In the former, the liquid solution obtained after the forming (e.g., metal-organic framework containing the first molecules has been separated) may undergo a second forming (e.g., a second metal-organic-framework crystallization or the like) using different reactant(s), conditions, etc. Such an approach can allow the extraction of remaining molecules. In an example, a method uses a mixture of polyester-nylon blend textiles. After extracting terephthalic acid, the leftover solution could undergo a second metal-organic-framework crystallization to extract the adipic acid or hexamethylenediamine using the aforementioned strategy.

In various examples, a method comprises a single step. In various examples, a single step comprises depolymerization and crystallization. In various examples, a single step comprises formation of a metal organic framework or frameworks in a single step. In various examples, a single step comprises allowing the processor to start on the polymer directly as the precursor for the metal-organic framework (MOF) formation (instead of monomers, which would require a separate depolymerization step at the beginning.)

In various examples, a reaction comprises one or more reactive crystallization(s). In various examples, a method recycles (or obtains one or more molecule(s) of interest from) a polymer/polymers (such as, for example, polyester(s) (such as, for example, poly(terephthalate)(s), poly(propylene terephthalate)(s), poly(butylene terephthalate)(s), polylactic acid(s), or the like), polyamide(s) (such as, for example, nylon(s) and the like), polyalcohol(s), polyurethane(s), polyurea(s), polycarbonate(s), polyether ether ketone(s), polyether ether ketone ketone(s), polyether ketone ketone(s), polyetherimide(s), polystyrene(s), or the like, or any combination thereof, lignins, starches, celluloses, or the like, or any combination thereof). In various examples, a method for recycling polymer(s) (or obtaining one or more molecule(s) of interest from) a polymer/polymers (such as, for example, polyester(s) (such as, for example, poly(terephthalate)(s), poly(propylene terephthalate)(s), poly(butylene terephthalate)(s), polylactic acid(s), or the like), polyamide(s) (such as, for example, nylon(s) and the like), polyalcohol(s), polyurethane(s), polyurea(s), polycarbonate(s), polyether ether ketone(s), polyether ether ketone ketone(s), polyether ketone ketone(s), polyetherimide(s), polystyrene(s), or the like, or any combination thereof, lignin(s), starche(s), cellulos(es), or the like, or any combination thereof) comprises reactive crystallization.

A method can use various monomers (such as, for example, impure monomers, structural analogs thereof, or the like). In various examples, monomer(s) is/are obtained from one or more polymer(s) described herein.

In various examples, impure monomer(s) (which may be recycled monomer(s) or the like) (or impure molecule(s) of interest) are terephthalic acid and adipic acid (which may obtained from depolymerization of polyester-nylon blend textiles or the like). After obtaining (e.g., isolating or the like) at least a portion of the isolating terephthalic acid, the remaining reaction mixture is subjected to a second forming reaction (e.g., formation of a second metal complex and/or metal-organic-framework and/or coordination polymer network or the like) and second decomposition to obtain (e.g., isolate or the like) at least a portion of the adipic acid.

In various examples, impure monomer(s) (which may be recycled monomer(s) or the like) (or impure molecule(s) of interest), structural analog(s) thereof, or the like, or any combination thereof comprise one or more impurit(ies) and the purified monomer(s) (which may be free monomer(s) or the like) (or purified molecule(s) of interest) and/or structural analog(s) thereof is/are substantially free or free of the impurit(ies) and/or the impure monomer(s) (which may be recycled monomer(s) or the like) (or impure molecule(s) of interest)) comprise one or more additive(s), structural analog(s) thereof, or the like, or any combination thereof and the purified monomer(s) (which may be free monomer(s) or the like) (or purified molecule(s) of interest) and/or structural analog(s) thereof is/are substantially free or free of the additive(s).

The presence (e.g., quantity) and/or absence of impurit(ies) and/or additive(s) can be determined by methods known in the art. In various examples, the presence (e.g., quantity) and/or absence of impurit(ies) and/or additive(s) is determined by spectroscopic method(s) (such, as for example, UV-VIS spectroscopy, IR spectroscopy, NMR spectroscopy (e.g., ¹H NMR spectroscopy, ¹³C NMR spectroscopy, or the like), spectrometric methods (e.g., mass spectrometry, GC-MS, LC-MS, or the like), liquid chromatography (such as, for example, high performance liquid chromatography (HPLC) or the like), thermogravimetric analysis (TGA), Platinum-Cobalt Color, acid number, inductively Coupled Plasma Optical Emission spectroscopy (ICP-OES), energy-dispersive x-ray spectroscopy (EDS), or the like, or any combination thereof. In various examples, the recycled monomer(s) comprise 5% or less, 2.5% or less, 1% or less, 0.1% or less. 0.05% or less by weight (based on the total weight of the recycled monomer(s) of impurit(ies) and/or additive(s)) as determined by one or more of the foregoing methods.

In various examples, the presence (e.g., quantity) and/or absence of impurit(ies) and/or additive(s) is below the detection limit of spectroscopic method(s) (such, as for example, UV-VIS spectroscopy, IR spectroscopy (e.g., FTIR or the like), NMR spectroscopy (e.g., ¹H NMR spectroscopy, ¹³C NMR spectroscopy, or the like), spectrometric methods (e.g., mass spectrometry, GC-MS, LC-MS, or the like), thermogravimetric analysis (TGA), acid number, or the like, or any combination thereof.

Metal complex(es), metal organic framework(s), coordination polymer network(s), or the like can be formed in various ways. In various examples, forming metal complex(es), metal organic framework(s), coordination polymer network(s), or the like, or any combination thereof comprises contacting impure monomer(s) (which may be recycled monomer(s) or the like) (or impure molecule(s) of interest), structural analog(s) thereof, or the like, or any combination thereof with one or more metal precursor(s) (e.g., in a reaction mixture or the like), where the metal complex(es), metal organic framework(s), coordination polymer network(s), or the like are formed. In various examples, the yield of a forming is 10% or higher. In various examples, forming one or more first metal complex(es), one or more first metal organic framework(s) (MOF(s)), one or more first coordination polymer network(s), or any combination thereof using (or from) a polymer-containing material (such as, for example, a textile, an article of manufacture, or the like, or any combination thereof) (e.g., in one pot and/or without using preformed (or isolated) impure monomer(s), structural analog(s) thereof, or any combination thereof.

In various examples, the impure monomer(s) and/or structural analog(s) thereof is/are formed from polymer-containing material or the like. In various examples, a polymer-containing material is a textile, an article of manufacture, or the like, or any combination thereof. In various examples, the impure monomer(s) and/or structural analog(s) thereof is/are formed from a textile, an article of manufacture, or the like, or any combination thereof.

In various examples, contacting impure monomer(s) (which may be recycled monomer(s) or the like) (or impure molecule(s) of interest), structural analog(s) thereof, or the like, or any combination thereof with one or more metal precursor(s) is carried out without preformed impure monomer(s) (which may be preformed recycled monomer(s) or the like) (or preformed impure molecule(s) of interest), structural analog(s) thereof, or the like, or any combination thereof (e.g., in a reaction mixture that does not comprise preformed impure monomer(s) (which may be preformed recycled monomer(s) or the like) (or preformed impure molecule(s) of interest), structural analog(s) thereof, or the like, or any combination thereof), where the metal complex(es), metal organic framework(s), coordination polymer network(s), or the like are formed.

Various metal precursors can be used. A metal precursor may be referred to as a metal-containing material. In various examples, a metal precursor is a salt or the like. In various examples, metal precursor(s) is/are chosen from metal salt(s), metal-containing clusters, metal particles (such as, for example, metal nanoparticles and the like), and the like, and structural analogs thereof, and any combination thereof. In various examples, the metal salt(s), independently, comprise one or more anion(s) chosen from F⁻, Cl⁻, Br⁻, I⁻, S⁻, CN⁻, NH₂⁻, OCN⁻, SCN⁻, C₂O₄²⁻, OH⁻, MnO₄⁻, PO₄³⁻, SO₄²⁻, NO₃⁻, NO₂⁻, ClO₄⁻, ClO₃⁻, ClO₂⁻, OCl⁻, IO₃⁻, BrO₃⁻, OBr⁻, CO₃²⁻, CrO₄²⁻, Cr₂O₇²⁻, CH₃COO⁻, HCOO⁻, and the like, and structural analogs thereof and/or one or more cation(s) chosen from transition metal cations (e.g., Sc, Ti, V, Cr, Fe, Mn, Co, Ni, Cu, Zn, Y, Zr, Nb, Mo, Ru, Rh, Ag, Zr, W cations, and the like), Group 13 cations (e.g., Al, Ga, In cations, and the like), alkaline earth cations (e.g., Mg, Ca, Sr, Ba cations, and the like), lanthanide cations(s) (e.g., hafnium and the like), and the like. In various examples, the metal precursor(s) is/are chosen from gallium nitrate (which may be a hydrate or anhydrous), gallium nanoparticles, gallium clusters, and the like, and any combination thereof.

Various amounts of metal precursor(s) can be used. In various examples, metal salt(s) is/are present (e.g., in a forming reaction mixture) at about 1 to about 20 wt %, including all 0.1 wt % values and ranges therebetween (e.g., about 3 to about 10 wt % or about 5 wt %), based on the total weight of a forming reaction mixture.

A forming may be carried out in a solvent or a mixture of solvents. In various examples, forming metal complex(es), metal organic framework(s) (MOF(s)), coordination polymer network(s), or the like is carried out in an aqueous solvent (e.g., water or the like). In various examples, forming metal complex(es), metal organic framework(s) (MOF(s)), coordination polymer network(s), or the like is carried out in solvent(s) chosen from water, organic solvents (e.g., dimethylformamide, dimethyl sulfoxide, N-methyl-2-pyrrolidone, cyrene, formic acid, acetone, or the like), ionic liquids, and the like, and any combination thereof. In various examples, a method comprises MOF formation under aqueous conditions, using a metal salt or salts and a polymer or polymers. In various examples, a method (e.g., polymer degradation, MOF formation, MOF decomposition, or any combination thereof, or a reaction mixture, or the like, or any combination thereof) is substantially free or free of organic solvents and/or ionic liquids.

Various amounts of solvent(s) may be used. In various examples, solvent(s) is/are present (e.g., in a forming reaction mixture) at about 50 to about 95 wt %, including all 0.1 wt % values and ranges therebetween (e.g., about 75 to about 90 wt % or about 90 wt %), based on the total weight of a forming reaction mixture.

A reaction forming metal complex(es), metal organic framework(s) (MOF(s)), coordination polymer network(s), or the like can be carried out under various reaction conditions.

A forming reaction can comprise one or more steps and each step can be performed under the same or different reaction conditions as other steps. A reaction can be carried out at various temperatures. In various examples, a reaction is carried out at about room temperature (e.g., from about 20 degrees Celsius (° C.) to about 22° C., including all 0.1° C. values and ranges therebetween) to about 600° C. (e.g., about 80° C. to about 600° C.), including all 0.1° C. values and ranges therebetween) above, or any combination thereof (e.g., where each reaction is performed at the same or different temperature as other steps). In various examples, a reaction is carried out at about 50° C. to about 400° C., including all 0.1° C. values and ranges therebetween (e.g., about 100° C. to about 300° C. or about 200° C. to about 250° C.

A forming reaction can be carried out at various pressures. In various examples, a reaction is carried out at below atmospheric pressure (e.g., about 1 bar), at about atmospheric pressure, or at greater than atmospheric pressure (e.g., heating in a sealed pressurized reaction vessel and the like). In various examples, a reaction is carried out at from about 1 bar to about 100 bar, including all 0.1 bar values and ranges therebetween, or any combination thereof (e.g., where each step is performed at a different pressure as other steps). In various examples, a reaction is carried out at from about 0.1 MPa to about 10 MPa, including all 0.1 MPa values and ranges therebetween (e.g., about 1 MPa to about 6 MPa or about 3 MPa. In various examples, each step is performed at the same or different pressure as other steps.

A forming reaction can be carried out for various times. Reaction time can depend on factors such as, for example, temperature, pressure, reaction component concentration(s), presence and/or intensity of an applied energy source, mixing (e.g., stirring or the like), or the like, or a combination thereof. In various examples, reaction time ranges from about minutes (e.g., 5 minutes) to about 3 days (or more), including all integer second values and ranges therebetween, or any combination thereof (e.g., where each step is performed at a different time as other steps). In various examples, reaction time is about one hour to about 3 days, including all integer second values and ranges therebetween (e.g., about 3 hours to about 9 hours or about 5 hours).

In various examples, a reaction is a hydrothermal reaction (e.g., carried out at about 210° C. and/or for about 5 hours or the like). In various examples, a method is carried out in air or an inert atmosphere.

In various examples, at least a portion of the, one or more, or all metal complex(es), metal organic framework(s), coordination polymer network(s), or the like are crystalline. In various examples, at least a portion or all metal complex(es), metal organic framework(s), coordination polymer network(s), or the like comprise a linear dimension or all linear dimensions of about 5 mm or less. In various examples, at least a portion of the, one or more, or all metal complex(es), metal organic framework(s), coordination polymer network(s), or the like are amorphous. In various examples, at least a portion of the, one or more, or all metal complex(es), metal organic framework(s), coordination polymer network(s), or the like are crystalline and/or at least a portion of one or more of the metal complex(es), metal organic framework(s), coordination polymer network(s), or the like are amorphous. In various examples, metal complex(es), metal organic framework(s), coordination polymer network(s), or the like are not amorphous.

In various examples, metal complex(es), metal organic framework(s) (MOF(s)), coordination polymer network(s), or the like are isolated from a forming reaction mixture. Non-limiting examples of isolation include filtration (e.g., vacuum filtration and the like), centrifugation, and the like.

Various impure monomers can used to form one or more metal complex(es), one or more metal organic framework(s) (MOF(s)), one or more coordination polymer network(s), or the like, or any combination thereof. In various examples, impure monomer(s) (which may be recycled monomer(s) or the like), structural analog(s) thereof, or any combination thereof (or molecules of interest) are, independently, ligands(s) of the metal complex(es) or linker(s) metal organic framework(s) or coordination polymer network(s), or the like.

In various examples, impure monomer(s) (which may be recycled monomer(s) or the like) (or impure molecule(s) of interest), structural analog(s) thereof, or the like, or any combination thereof have a purity of 99.9% or less, 99% or less, or 90% or less. In various examples, the impure monomer(s) (which may be recycled monomer(s) or the like) (or impure molecule(s) of interest), structural analog(s) thereof, or the like, or any combination thereof comprise impurit(ies) and/or additive(es) 0.1 to 15 wt %, including all 0.1 wt % values and ranges therebetween, based on the total weight of impure monomer(s) and/or structural analog(s) and impurit(ies) and/or additive(es). In various examples, impure monomer(s) comprise less than or at least 1 weight percent (wt %) impurit(ies) and/or additive(es). Non-limiting examples of impurities include impurities typically found in commercial polymers, articles of manufacture comprising commercial polymers, and the like (such as, for example, pigments, dyes, and the like, and any combination thereof). Non-limiting examples of additives include additives typically found in commercial polymers, articles of manufacture comprising commercial polymers, and the like (such as, for example, surface additives, plasticizers, and the like, and any combination thereof).

In various examples, impure monomer(s) (which may be recycled monomer(s) or the like) (or impure molecule(s) of interest) and/or structural analog(s) thereof exhibit an absorbance at one or more visible wavelength(s) (e.g., about 380 nm to about 700 nm, including all 0.1 nm values and ranges therebetween) of about 0.01 to about 1.5, including all 0.01 values and ranges therebetween, measured at a impure monomer(s) and/or structural analog(s) concentration of about 1 micromolar to about 200 millimolar (e.g., about 1 millimolar or 10 millimolar), including all 0.1 microlmolar values and ranges therebetween, and path length of about 1 centimeter (cm).

Various amounts of impure monomer(s), structural analog(s) thereof, or the like, or any combination thereof can be used. In various examples, impure monomer(s) is/are present (e.g., in a forming reaction mixture) at about 1 to about 20 wt %, including all 0.1 wt % values and ranges therebetween (e.g., about 1 to about 10 wt % or about 3 wt %), based on the total weight of a forming reaction mixture.

Various MOFs can be formed. In various examples, metal organic framework(s) are chosen from gallium MOFs (e.g., MIL-53 (Ga) and the like), UiO-66(Zr), UiO-66(Hf), MIL-53(Al), MIL-47(V), Cr-MOF20, MIL-47(V), MIL-53(Cr), MIL-53(Fe), MIL-53(Ga), MIL-101(Cr), MIL-101(Fe), MOF-2, MOF-5, and copper terephthalates, and the like, and any combination thereof. In various examples, the MOF(s), independently (in the case of combinations of MOFs), have a linear dimension (e.g., a diameter or the like), which may be a longest linear dimension, of about 0.1 micrometers to about 100 micrometers, including all 0.01 micrometer values and ranges therebetween.

In various examples, a method further comprises providing impure monomer(s) (which may be recycled monomer(s) or the like) (or impure molecule(s) of interest), structural analog(s) thereof, or the like, or any combination thereof. In various examples, impure monomer(s), structural analog(s) thereof, or the like, or any combination thereof are provided prior to forming metal complex(es), metal organic framework(s) (MOF(s)), coordination polymer network(s), or the combination thereof. In various examples, impure monomer(s) (which may be recycled monomer(s) or the like), structural analog(s) thereof, or the like, or any combination thereof is/are provided by degradation (e.g., depolymerization or the like) of polymer(s). In various examples, one or more impure monomer(s) (which may be recycled monomer(s) or the like), structural analog(s) thereof, or the like, or any combination thereof is/are provided by solvolysis (such as, for example, a hydrolysis, methanolysis, glycolysis, or the like) of one or more polymer(s) (e.g., alkaline hydrolysis, acid hydrolysis, glycolysis, alcoholysis, aminolysis, or the like, or any combination thereof).

Various polymers can be degraded (e.g., depolymerized or the like). In various examples, a polymer comprises one or more structural unit(s) (e.g., first structural unit(s), second structural unit(s), etc.) (or repeat unit(s) or the like) that are independently formed from polymerization of one or more or all of the purified monomer(s), structural analog(s) thereof, or the like or any combination thereof.

In various examples, impure monomer(s) (which may be recycled monomer(s) or the like), structural analog(s) thereof, or the like, or any combination thereof is/are provided by solvolysis (such as, for example, a hydrolysis reaction, methanolysis reaction, glycolysis reaction, or the like) or the like of one or more polymer(s). In various examples, solvolysis (such as, for example, a hydrolysis, methanolysis, glycolysis, or the like) of one or more polymer(s) comprises forming a mixture comprising one or more base(s) and one or more polymer(s). A solvolysis reaction (such as, for example, a hydrolysis reaction, methanolysis reaction, glycolysis reaction, or the like) can be performed under various reaction conditions.

Various bases or acids may be used in a solvolysis reaction (such as, for example, a hydrolysis reaction, methanolysis reaction, glycolysis reaction, or the like). Combinations of bases or acids can be used. Non-limiting examples of basis include lithium hydroxide, sodium hydroxide, potassium hydroxide, rubidium hydroxide, cesium hydroxide, barium hydroxide, sodium carbonate, potassium carbonate, and the like, and any combination thereof. Non-limiting examples of acids include mineral acids (such as, for example, sulfuric acid, nitric acid, hydrochloric acid, hydrobromic acid, and the like) and organic acids (such as, for example, acetic acid, trifluoroacetic acid, formic acid, and the like), and the like, and any combination thereof.

Various amounts of base(s) or acid(s) can be used in a solvolysis reaction (such as, for example, a hydrolysis reaction, methanolysis reaction, glycolysis reaction, or the like). In various examples, base(s) or acid(s) is/are present (e.g., in the hydrolysis reaction mixture) at about 1 to about 20 wt %, including all 0.1 wt % values and ranges therebetween (e.g., about 3 to about 10 wt % or about 5 wt %), based on the total weight of the solvolysis reaction mixture.

In various examples, a polymer or polymers (or a polymer-containing material or materials) is/are present in a raw material. In various examples, a raw material comprises (or is) a textile, an article of manufacture (such as, for example, a container (such as, for example, a bottle, a food trays, a food packaging, or the like), a rug/carpet, a structural element, a surface element (such as, for example, a component or part in an automobile, a plane, or the like), or the like, or any combination thereof. In various examples, a raw material comprises (or is) a waste textile, a waste article of manufacture (such as, for example, a waste container (such as, for example, a waste bottle, a waste food tray, a waste food packaging, or the like), a waste rug/carpet, a waste structural element, a waste surface element (such as, for example, a component or part in an automobile, a plane, or the like), or the like, or any combination thereof.

Various amounts of polymer(s) (e.g., raw material(s), which may be waste material(s)) can be used. In various examples, polymer(s) (e.g., raw material(s)) is/are present (e.g., in a hydrolysis reaction mixture) at about 1 to about 50 wt %, including all 0.1 wt % values and ranges therebetween (e.g., about 1 to about 20 wt % or about 1 wt %), based on the total weight of the hydrolysis reaction mixture.

A solvolysis reaction (such as, for example, a hydrolysis reaction, methanolysis reaction, glycolysis reaction, or the like) may be carried out in a solvent or a mixture of solvents. In various examples, a solvolysis reaction (such as, for example, a hydrolysis reaction, methanolysis reaction, glycolysis reaction, or the like) is carried out in an aqueous solvent (e.g., water or the like). In various examples, a (such as, for example, a hydrolysis reaction, methanolysis reaction, glycolysis reaction, or the like) is carried out in solvent(s) chosen from water, organic solvents (e.g., alcohols, glycols, dimethylformamide, dimethyl sulfoxide, N-methyl-2-pyrrolidone, cyrene, formic acid, acetone, or the like), ionic liquids, and the like, and any combination thereof.

Various amounts of solvent(s) may be used. In various examples, solvent(s) is/are present (e.g., in a hydrolysis reaction mixture) at about 50 to about 95 wt %, including all 0.1 wt % values and ranges therebetween (e.g., about 75 to about 95 wt % or about 95 wt %), based on the total weight of the hydrolysis reaction mixture.

A solvolysis reaction (such as, for example, a hydrolysis reaction, methanolysis reaction, glycolysis reaction, or the like) can be carried out at various temperatures. In various examples, a solvolysis reaction is carried out at about 50° C. to about 100° C., including all 0.1° C. values and ranges therebetween (e.g., about 80° C. to about 100° C. or about 90° C.).

A solvolysis reaction (such as, for example, a hydrolysis reaction, methanolysis reaction, glycolysis reaction, or the like) can be carried out at various pressures. In various examples, a solvolysis reaction is carried out at from about 0.1 MPa to about 1 MPa, including all 0.1 MPa values and ranges therebetween (e.g., about 0.1 MPa to about 0.5 MPa or about 0.1 MPa.

A solvolysis reaction (such as, for example, a hydrolysis reaction, methanolysis reaction, glycolysis reaction, or the like) can be carried out for various times. Reaction time can depend on factors such as, for example, temperature, pressure, reaction component concentration(s), presence and/or intensity of an applied energy source, mixing (e.g., stirring or the like), or the like, or a combination thereof. In various examples, reaction time is about one hour to about 12 hours, including all integer second values and ranges therebetween (e.g., about 2 hours to about 8 hours or about 3 hours).

In various examples, polymer(s) is/are chosen from organic polymers, biopolymers, and the like, and any combination thereof. In various examples, an organic polymer is a polyester (such as, for example, a poly(terephthalate), a poly(propylene terephthalate), a poly(butylene terephthalate), a polylactic acid, or the like), a polyamide (such as, for example, a nylon or the like), a polyalcohol, a polyurethane, a polyurea, a polycarbonate, a polyether ether ketone, a polyether ether ketone ketone, a polyether ketone ketone, a polyetherimide, a polystyrene, or the like, or any combination thereof. In various examples, a biopolymer is a lignin, a starch, a cellulose, or the like, or any combination thereof.

In various examples, impure monomer(s) (which may be recycled monomer(s) or the like) (or impure molecule(s) of interest), structural analog(s) thereof, or the like, or any combination thereof comprise (or is/are) organic acid(s), polyol(s), amine(s) (which may be diamines or the like), any of which may, independently, are protonated or deprotonated, or the like, or any combination thereof. In various examples, impure monomer(s) (which may be recycled monomer(s) or the like) (or impure molecule(s) of interest), structural analog(s) thereof, or the like, or any combination thereof and/or free monomer(s) (or purified molecule(s) of interest) comprise one or more organic acid(s), one or more polyol(s), one or more amine(s), or structural analog(s) thereof is/are, independently, a deprotonated (e.g., a salt) or protonated structural analog of the organic acid(s), polyol(s), amine(s), or any combination thereof. In various examples, the organic acid(s) is/are chosen from terephthalic acid, isophthalic acid, phthalic acid, adipic acid, and the like, and structural analogs thereof, and any combination thereof. In various examples, the polyol (s) is/are chosen from diols (such as, for example, ethylene glycol, propane diol, butanediol, and the like), and the like, and structural analogs thereof, and any combination thereof. In various examples, the organic amine(s) is/are chosen from diamines (such as, for example, methylenediamine, hexamethyldiamine, and the like), and the like, and structural analogs thereof, and any combination thereof.

In various examples, impure monomer(s) (which may be recycled monomer(s) or the like), structural analog(s) thereof, or the like, or any combination thereof is/are formed from a textile, an article of manufacture (such as, for example, a container (such as, for example, a bottle, a food trays, a food packaging, or the like), a rug/carpet, a structural element, a surface element (such as, for example, a component or part in an automobile, a plane, or the like), or the like, or any combination thereof.

In various examples, forming one or more metal complex(es), one or more metal organic framework(s), one or more coordination polymer network(s), or any combination thereof using one or more impure monomer(s), structural analog(s) thereof, or any combination thereof comprises: mixing one or more metal salt(s), one or more polymer-containing material(s), where each of the polymer-containing material(s) independently comprises the one or more polymer(s), and one or more impurities, and optionally an aqueous solvent to form a mixture; and reacting the mixture in thermal degradation condition(s) to form a thermal degraded or a hydrolyzed mixture, such that the polymer(s) in the polymer-containing material(s) depolymerize(s) to form or release one or more monomer(s) mixed with one or more impurities forming one or more impure monomer(s), where the one or more monomer(s) react with the metal salt(s) to form the one or more metal complex(es), the one or more metal organic framework(s), the one or more coordination polymer network(s), or the combination thereof, and the one or more impurit(ies) is/are substantially excluded from the one or more metal complex(es), the one or more metal organic framework(s), the one or more coordination polymer network(s), or the combination thereof. In various examples, the thermal degradation condition(s) comprise(s) a temperature of about 100° C. to about 300° C., including all 0.1° C. values and ranges therebetween, and optionally a water- or steam-containing environment or the like. In various examples, a reaction time of reacting the mixture in the thermal degradation condition(s) is about 1 hour to about 3 days, including all 0.1 minute values and ranges therebetween. In various examples, depolymerizing the polymer to release one or more monomer(s) and reacting the released one or more monomer(s) with the metal salt(s) is/are in one pot, such that the one or more monomer(s) is/are substantially converted to the one or more metal complex(es), the one or more metal organic framework(s), the one or more coordination polymer network(s), or the combination thereof without keeping accumulated in the thermal degraded or the hydrolyzed mixture comprising the metal salt(s). In various examples, a method further comprises: washing the thermal degraded or the hydrolyzed mixture or one or more crystalline material(s) independently comprising the one or more metal complex(es), the one or more metal organic framework(s), the one or more coordination polymer network(s), or the combination thereof in the thermal degraded or the hydrolyzed mixture with one or more solvent(s) to remove the one or more impurit(ies) and obtain a purified metal complex(es), metal organic framework(s), coordination polymer network(s), or any combination thereof; and decomposing the purified metal complex(es), the metal organic framework(s), coordination polymer network(s), or the combination thereof to produce a purified monomer(s) and/or structural analog(s) thereof substantially free of the one or more impurities.

In various examples, a method is carried out using a single reaction mixture and/or without isolation of impure monomer(s), structural analog(s) thereof, or any combination thereof. In various examples, providing impure monomer(s), structural analog(s) thereof, or any combination thereof and forming metal complex(es), first metal organic framework(s) (MOF(s)), coordination polymer network(s), or any combination thereof are carried out in a single reaction mixture and/or without isolation of impure monomer(s), structural analog(s) thereof, or any combination thereof. In various examples, a single reaction mixture comprises one or more metal salt(s), one or more polymer-containing material, where each of the polymer-containing material(s) independently comprises one or more polymer(s) and one or more impurities, and optionally an aqueous solvent or steam, and where the polymer-containing material(s) is/are chosen from textiles, articles of manufacture, and the like, and any combination thereof.

In various examples, a method comprises (or further comprises) isolating (e.g., a portion, substantially all, or all of the) metal complex(es), metal organic framework(s) (MOF(s)), coordination polymer network(s), or the like, or any combination thereof. Metal complex(es), metal organic framework(s) (MOF(s)), coordination polymer network(s), or the like can be isolated by methods known in the art.

In various examples, a method comprises (or further comprises) contacting (such as, for example, washing or the like) metal complex(es), metal organic framework(s) (MOF(s)), coordination polymer network(s), or the like, or any combination thereof (which may be isolated) with one or more solvent(s) (examples of which are disclosed herein with respect to formation of) metal complex(es), metal organic framework(s) (MOF(s)), coordination polymer network(s), or the like, or any combination thereof). In various examples, contacting repeated a desired number of times.

In various examples, a method comprises (or further comprises) decomposing (e.g., a portion, substantially all, or all of the) metal complex(es), metal organic framework(s), coordination polymer network(s), or the like, where purified monomer(s) (e.g., free monomer(s) or the like) (or purified molecule(s) of interest) and/or structural analog(s) thereof are formed.

In various examples, decomposing (e.g., a portion, substantially all, or all of the) metal complex(es), metal organic framework(s), coordination polymer network(s), or the like comprises contacting (e.g., forming a mixture (which may be a solution or the like) or the like with) metal complex(es), metal organic framework(s), coordination polymer network(s), or the like with one or more acid(s), one or more base(s), optionally, one or more organic solvent(s), one or more ionic liquid(s), or the like, or any combination thereof. In various examples, decomposing (e.g., a portion, substantially all, or all of the) metal complex(es), metal organic framework(s), coordination polymer network(s), or the like comprises contacting the metal complex(es), metal organic framework(s), coordination polymer network(s), or the like with one or more acid(s) forms metal precursor(s), structural analog(s) thereof, or the like. In various examples, decomposing comprises heating the metal complex(es), the metal organic framework(s), coordination polymer network(s), or the like (e.g., heating at about 100° C. in a mineral acid (such as, for example, nitric acid or the like) for about 6 hours).

Various amounts of MOF(s) can be used. In various examples, MOF(s) is/are present (e.g., in a decomposition reaction mixture) at about 1 to about 15 wt %, including all 0.1 wt % values and ranges therebetween (e.g., about 5 to about 15 wt % or about 15 wt %), based on the total weight of the reaction mixture.

In various examples, a decomposing is carried out in the absence of added solvent(s). In various examples, decomposing is carried out in one or more solvent(s). Non-limiting examples of solvents include aqueous solvents (e.g., water, acids, bases, and the like), organic solvents (e.g., dimethylformamide, dimethyl sulfoxide, N-methyl-2-pyrrolidone, cyrene, formic acid, acetone, or the like), ionic liquids, and the like.

In various examples, decomposing is carried out in presence of one or more additive(s). Non-limiting examples of additives include acids, bases, and the like. Non-limiting examples of acids include nitric acid, sulfuric acid, phosphoric acid, hydrochloric acid, perchloric acid, and the like, and any combination thereof. Non-limiting examples of bases include sodium hydroxide, potassium hydroxide, lithium hydroxide, calcium hydroxide, barium hydroxide, magnesium hydroxide, ammonium hydroxide, tetramethylammonium hydroxide, sodium carbonate, potassium carbonate, trisodium phosphate, tripotassium phosphate, and the like, and any combination thereof.

Various amounts of acid(s) or base(s) can be used. In various examples, acid(s) or base(s) is/are present (e.g., in decomposition reaction mixture) at about 85 to about 98 wt %, including all 0.1 wt % values and ranges therebetween (e.g., about 85 to about 90 wt % or about 85 wt %), based on the total weight of a decomposition reaction mixture.

Decomposing can be carried out at various pH values. In various examples, decomposing is carried out at (or a decomposition reaction mixture comprises) a pH of 0-14, including all 0.1 pH units and ranges therebetween.

A decomposing metal complex(es), metal organic framework(s) (MOF(s)), coordination polymer network(s), or the like can be performed under various reaction conditions. A decomposing reaction can comprise one or more steps and each step can be performed under the same or different reaction conditions as other steps.

A decomposing reaction can be carried out at various temperatures. In various examples, a decomposing reaction is carried out without exogenous heating or with exogenous heating. In various examples, a decomposing reaction is carried out at about room temperature (e.g., from about 20° C. to about 22° C., including all 0.1° C. values and ranges therebetween), or above room temperature (e.g., above room temperature up to or about a boiling point of the solvent(s), if present) (e.g., from above room temperature to about 600° C. or above, or any combination thereof (e.g., where each decomposing reaction is performed at the same or a different temperature as other steps). In various examples, a decomposing is carried out at or about the boiling point of the acid(s) (e.g., mineral acid(s) or the like), base(s), organic solvent(s), or the like. In various examples, a decomposing reaction is carried out at about 25° C. to about 100° C., including all 0.1° C. values and ranges therebetween (e.g., about 50° C. to about 100° C. or about 100° C.).

A decomposing reaction can be carried out at various pressures. In various examples, a decomposing reaction is carried out at below atmospheric pressure (e.g., about 1 bar), at about atmospheric pressure, or at greater than atmospheric pressure (e.g., heating in a sealed pressurized reaction vessel and the like). In various examples, a decomposing reaction is carried out at from about 1 bar to about 100 bar, including all 0.1 bar values and ranges therebetween, or any combination thereof (e.g., where each step is performed at a different pressure as other steps). In various examples, a reaction is carried out at from about 0.1 MPa to about 1 MPa, including all 0.1 MPa values and ranges therebetween (e.g., about 1 MPa to about 0.5 MPa or about 0.1 MPa.

A decomposing reaction can be carried out for various times. Reaction time can depend on factors such as, for example, temperature, pressure, reaction component concentration(s), presence and/or intensity of an applied energy source, mixing (e.g., stirring or the like), or the like, or a combination thereof. In various examples, reaction times range from about minutes (e.g., 5 minutes) to about 3 days (or more), including all integer second values and ranges therebetween, or any combination thereof (e.g., where each step is performed at a different time as other steps). In various examples, decomposition reaction time is about one hour to about 24 hours, including all integer second values and ranges therebetween (e.g., about 1 hour to about 8 hours or about 6 hours).

In various examples, a method forms purified monomers and/or structural analog(s) thereof. In various examples, at least a portion or all of the purified monomer(s) (which may be free monomer(s) or the like) (or purified molecule(s) of interest) and/or structural analog(s) thereof is/are substantially colorless or colorless. The presence (e.g., quantity) and/or absence of color can be determined by methods known in the art. In various examples, the presence (e.g., quantity) and/or absence of color is determined by spectroscopic method(s) (such, as for example, UV-VIS spectroscopy or the like, or any combination thereof), Platinum-Cobalt Color, acid number, or the like, or any combination thereof. In various examples, the purified monomer(s) do not have detectible absorbance and/or 98% or greater, 99% or greater, 99.5% or greater, 99.9% or greater or substantially 100% transmittance of visible light wavelengths (e.g., about 400 nanometers to about 700 nanometers).

In various examples, purified monomer(s) (which may be free monomer(s)) and/or structural analog(s) thereof (or purified molecule(s) of interest (e.g., terephthalic acid or the like or any combination thereof) has higher purity than the impure monomer(s) (which may be a recycled monomer) (or impure molecule(s) of interest), structural analog(s) thereof, or the like, or any combination thereof used to produce the purified monomer(s) (which may be free monomer(s)) (or purified molecule(s) of interest) and/or structural analog(s) thereof. In various examples, purified monomer(s) (or purified molecule(s) of interest) purity level exhibit(s) high-purity appearance (such as, for example, white-transparent powders in the case of terephthalic acid). In various examples, purified monomer(s) (which may be free monomer(s)(or purified molecule(s) of interest) and/or structural analog(s) thereof purity level exhibit(s) substantially the same or the same thermogravimetric or spectroscopic features as the corresponding analytical-grade molecules. In various examples, purified monomer(s) (which may be free monomer(s) or purified molecule(s) of interest) and/or structural analog(s) exhibit(s) a transparent color profile (e.g., that shows no features substantially different or different than the same monomer(s) signal(s) when tested using liquid chromatography or the like).

In various examples, a method further comprises isolating (e.g., a portion, substantially all, or all of the) purified monomer(s) (or purified molecule(s) of interest) and/or the structural analog(s) thereof. In various examples, purified monomer(s) (e.g., free monomer(s) or the like) (or purified molecule(s) of interest) and/or structural analog(s) thereof is/are isolated from the decomposition reaction mixture. Non-limiting examples of isolation include filtration (e.g., vacuum filtration and the like), centrifugation, and the like, which may remove substantially all or all of the solid(s) from the decomposition reaction mixture. Other non-limiting examples of isolation include distillation (e.g., vacuum distillation and the like, and the like, which may remove substantially all or all of the purified monomer(s) (e.g., free monomer(s) or the like) (or purified molecule(s) of interest) from the decomposition reaction mixture.

In various examples, in the case where not all impure monomer(s) (which may be recycled monomer(s) or the like) (or impure molecule(s) of interest), structural analog(s) thereof, or the like, or any combination thereof are reacted in the forming reaction and/or decomposing reactions, the reaction mixture remaining after isolation of the purified monomer(s) (which may be free monomer(s) or the like) (or impure molecule(s) of interest) and/or structural analog(s) is subjected to additional/subsequent forming and/or decomposing reaction(s). In various examples, a method further comprises a second forming and/or second decomposing. In various examples, one or more or all of the additional forming and/or decomposing reaction(s) (e.g., the second forming and/or the second decomposing or the like) are carried out under the same conditions as the initial (e.g., first) forming and/or initial (e.g., first) decomposing. In various examples, one or more of all of the additional forming and/or decomposing reaction(s) (e.g., the second forming and/or the second decomposing or the like) are carried out under different conditions (e.g., different reactant(s), different reactant concentration(s), different reaction time and/or temperature, or the like, or any combination thereof) from the initial (e.g., first) forming and/or initial (e.g., first) decomposing or two or more or all of the other additional/subsequent forming and/or decomposing reaction(s). Forming and/or decomposing can be carried out a desired number of times.

In various examples, a method comprises (or further comprises) use of purified monomer(s) (which may be free monomer(s)), structural analog(s) thereof, or the like, or any combination thereof in a polymerization reaction. In various examples, polymerization forms a polymer described herein. In various examples, polymerization forms a polyester (such as, for example, a poly(terephthalate), a poly(propylene terephthalate), a poly(butylene terephthalate), a polylactic acid, or the like), a polyamide (such as, for example, a nylon or the like), a polyalcohol, a polyurethane, a polyurea, a polycarbonate, a polyether ether ketone, a polyether ether ketone ketone, a polyether ketone ketone, a polyetherimide, a polystyrene, or the like, or any combination thereof.

In various examples, one or more or all purified monomer(s) and/or structural analog(s) thereof is/are subjected to one or more purification(s) (which are not a purification method of the present disclosure) prior to use in a polymerization reaction. Non-limiting example of purification include solvent extraction, distillation, crystallization, trituration, chromatography, and the like, and any combination thereof.

In various examples, a method comprises (or further comprises) isolation (such as, for example, recycling or the like) of at least a portion of or substantially all (or all) of the acid(s) or base(s) or metal ions (of the metal precursor(s), which may be in the form of metal salt(s)), or both used to form the metal complex(es), the MOF(s), the coordination polymer network(s), or the like, or any combination thereof. In various examples, at least a portion of or substantially all (or all) of the acid(s) or base(s) are isolated by distillation or the like and/or at least a portion of or substantially all (or all) of the metal ion(s) (such as, for example, metal salt(s) or the like) are isolated by solvent removal (such as, for example, evaporation, distillation, or the like. In various examples, the isolated acid(s) or base(s) and/or isolated metal precursor(s) used in a subsequent method of the present disclosure (e.g., are recycled).

In various examples, a method comprises (or further comprises) isolation (such as, for example, recycling or the like) of at least a portion of or substantially all (or all) of the acid(s) or base(s) or metal ions (of the metal precursor(s), which may be in the form of metal salt(s)), or both used to form the metal complex(es), the MOF(s), the coordination polymer network(s), or the like, or any combination thereof. In various examples, at least a portion of or substantially all (or all) of the acid(s) or base(s) are isolated by distillation or the like and/or at least a portion of or substantially all (or all) of the metal ion(s) (such as, for example, metal salt(s) or the like) are isolated by solvent removal (such as, for example, evaporation, distillation, or the like. In various examples, the isolated acid(s) or base(s) and/or isolated metal precursor(s) used in a subsequent method of the present disclosure (e.g., are recycled).

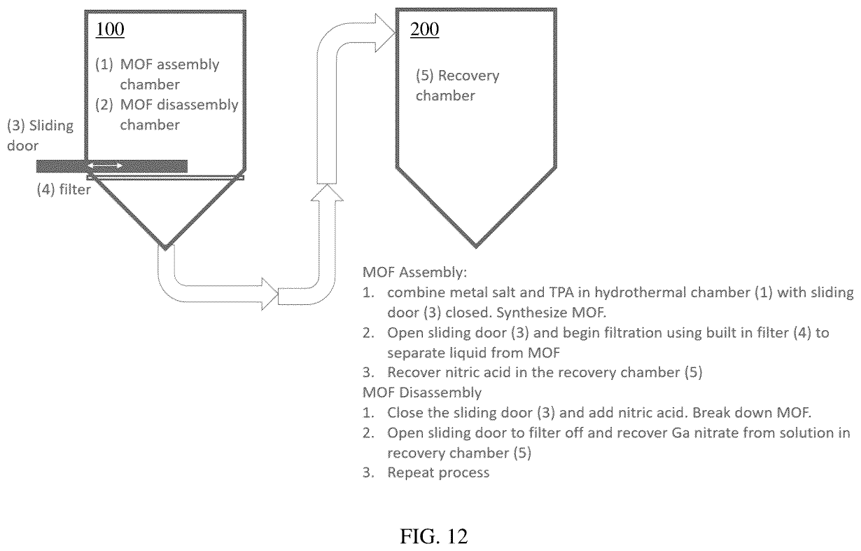

In various examples, a method or a portion thereof is carried out in a batch process, a continuous process, a semi-continuous process, or the like, or any combination thereof. In various examples, a method or a portion thereof is carried out in a system. An example of a system is shown in FIG. 12. In the system of FIG. 12, a first chamber 100 is configured to carry out formation of one or more metal complex(es), one or more metal organic framework(s) (MOF(s)), one or more coordination polymer network(s), or the like, or any combination thereof and isolation of same and disassembly of same. A second chamber 200 is configured to recover metal salt(s) formed by the disassembly process. In various examples, a first chamber and a second chamber are in fluid connection.

In an aspect, the present disclosure provides methods of making one or more metal complex(es), one or more metal organic framework(s) (MOF(s)), one or more coordination polymer network(s), or the like, or any combination thereof. Non-limiting examples of the methods are disclosed herein.

In various examples, a method of making one or more metal complex(es), one or more metal organic framework(s) (MOF(s)), one or more coordination polymer network(s), or the like, or any combination thereof is as described herein. In various examples, a method of making one or more metal complex(es), one or more metal organic framework(s) (MOF(s)), one or more coordination polymer network(s), or the like, or any combination thereof comprises: forming one or more metal complex(es), one or more metal organic framework(s) (MOF(s)), one or more coordination polymer network(s), or the like, or any combination thereof comprising one or more impure monomer(s) (which may be recycled monomer(s) or the like) (or impure molecule(s) of interest), structural analog(s) thereof, or any combination thereof; and optionally, isolating the metal complex(es), one or more metal organic framework(s) (MOF(s)), coordination polymer network(s), or the like.

In an aspect, the present disclosure provides uses of purified monomer(s) (or purified molecule(s) of interest) and/or structural analog(s) thereof. In various examples, purified monomer(s) (or purified molecule(s) of interest), structural analog(s) thereof, or the like, or any combination thereof are made by a method of the present disclosure. Non-limiting examples of uses of purified monomer(s) (or purified molecule(s) of interest) and structural analog(s) thereof are disclosed herein.

Purified monomer(s) or structural analog(s) thereof can be used in various polymerization reactions and/or to produce various polymers. In various examples, a polymer is a homopolymer, a copolymer, or the like. In various examples, a polymer comprises one or more polymerized purified monomer(s) (or purified molecule(s) of interest) and/or structural analog(s) thereof. In various examples, a polymer comprises structural units formed from one or more monomer(s) (such as, for example, purified monomer(s) (or purified molecule(s) of interest) and/or structural analog(s) thereof)), where each monomer is independently obtained from another polymer (e.g., by a method of the present disclosure). Non-limiting examples of polymerization reactions and polymers are described herein.

In various examples, a polymer is formed from at least one purified monomer(s) (or purified molecule(s) of interest) and/or structural analog(s) thereof (e.g., a monomer or monomers produced by a method of the present disclosure). In various examples, a polymer is formed from two or more purified monomers (or purified molecules of interest) and/or structural analog(s) thereof (e.g., monomers produced by a method of the present disclosure). In various examples, a polymer further comprises one or more monomer(s) that are not purified monomer(s) (e.g., a monomer or monomer(s) not produced by a method of the present disclosure).

In various examples, a polymer comprises (or is chosen from) organic polymers, biopolymers, and the like, and any combination thereof. In various examples, an organic polymer is a polyester (such as, for example, a poly(terephthalate), a poly(ethylene terephthalate), a poly(propylene terephthalate), a poly(butylene terephthalate), a polylactic acid, or the like), a polyamide (such as, for example, a nylon or the like), a polyalcohol, a polyurethane, a polyurea, a polycarbonate, a polyether ether ketone, a polyether ether ketone ketone, a polyether ketone ketone, a polyetherimide, a polystyrene, or the like, or any combination thereof. In various examples, a biopolymer is a lignin, a starch, a cellulose, or the like, or any combination thereof.

In various examples, purified monomer(s) (or purified molecule(s) of interest) and/or structural analog(s) thereof (e.g., a monomer or monomers produced by a method of the present disclosure) is/are chosen from organic acid(s), polyol(s), amine(s) (which may be diamines or the like), any of which may, independently, are protonated or deprotonated, or the like, or any combination thereof. In various examples, organic acid(s) is/are chosen from terephthalic acid, isophthalic acid, phthalic acid, adipic acid, and the like, and structural analogs thereof, and any combination thereof. In various examples, polyol (s) is/are chosen from diols (such as, for example, ethylene glycol, propane diol, butanediol, and the like), and the like, and structural analogs thereof, and any combination thereof. In various examples, organic amine(s) is/are chosen from diamines (such as, for example, methylenediamine, hexamethyldiamine, and the like), and the like, and structural analogs thereof, and any combination thereof.

In various examples, a polymer does not comprise one or more impurit(ies) and/or additive(s) present in the impure monomer(s), structural analog(s) thereof, or the combination thereof from which purified monomer(s) and/or structural analog(s) was/were formed. In various examples, a polymer is substantially free or free of the impurit(ies) and/or additive(s) present in the impure monomer(s), structural analog(s) thereof, or the combination thereof from which purified monomer(s) and/or structural analog(s) was/were formed.

The presence (e.g., quantity) and/or absence of impurit(ies) and/or additive(s) in a polymer can be determined by methods known in the art. In various examples, the presence (e.g., quantity) and/or absence of impurit(ies) and/or additive(s) is determined by spectroscopic method(s) (such, as for example, UV-VIS spectroscopy, IR spectroscopy, NMR spectroscopy (e.g., ¹H NMR spectroscopy, ¹³C NMR spectroscopy, or the like), spectrometric methods (e.g., mass spectrometry, gas chromatography-mass spectrometry (GC-MS), liquid chromatography-mass spectrometry (LC-MS), or the like), liquid chromatography (such as, for example, high performance liquid chromatography (HPLC) or the like), thermogravimetric analysis (TGA), Platinum-Cobalt Color, acid number, inductively Coupled Plasma Optical Emission spectroscopy (ICP-OES), energy-dispersive x-ray spectroscopy (EDS), or the like, or any combination thereof. In various examples, a polymer comprises 5% or less, 2.5% or less, 1% or less, 0.1% or less. 0.05% or less by weight (based on the total weight of the polymer, impurit(ies) and/or additive(s)) as determined by one or more of the foregoing methods.

In various examples, the presence (e.g., quantity) and/or absence of impurit(ies) and/or additive(s) in a polymer is below the detection limit of spectroscopic method(s) (such, as for example, UV-VIS spectroscopy, IR spectroscopy (e.g., FTIR or the like), NMR spectroscopy (e.g., ¹H NMR spectroscopy, ¹³C NMR spectroscopy, or the like), spectrometric methods (e.g., mass spectrometry, GC-MS, LC-MS, or the like), thermogravimetric analysis (TGA), acid number, or the like, or any combination thereof.

Purified monomer(s) and/or structural analog(s) thereof (e.g., polymer(s) produced using the purified monomer(s) and/or the structural analog(s) thereof) can be used to form various articles of manufacture. Non-limiting examples of articles of manufacture (e.g., comprising one or more polymer(s) produced using the purified monomer(s) or the structural analog(s) thereof of the present disclosure) are described herein.

The following Statements provide examples of methods and polymers of the present disclosure:

Statement 1. A method of recycling one or more polymer(s) (e.g., purifying recycled monomer(s) obtained from polymer(s)) (or obtaining one or more molecule(s) of interest (e.g., from a polymer/polymers or the like)) comprising forming one or more metal complex(es), one or more metal organic framework(s) (MOF(s)), one or more coordination polymer network(s), or the like, or any combination thereof comprising one or more impure monomer(s) (which may be recycled monomer(s) or the like) (or impure molecule(s) of interest), structural analog(s) thereof, or any combination thereof; optionally, isolating (e.g., a portion, substantially all, or all of) the metal complex(es), one or more metal organic framework(s) (MOF(s)), coordination polymer network(s), or the like, or the combination thereof; optionally, decomposing (e.g., a portion, substantially all, or all of) the metal complex(es), metal organic framework(s), coordination polymer network(s), or the like, or the combination thereof where purified monomer(s) (e.g., free monomer(s) or the like) (or purified molecule(s) of interest) and/or structural analog(s) thereof are formed; and optionally, isolating the purified monomer(s) (or purified molecule(s) of interest) and/or the structural analog(s) thereof.

Statement 2. A method according to Statement 1, the method further comprising providing impure monomer(s) (which may be recycled monomer(s) or the like) (or impure molecule(s) of interest), structural analog(s) thereof, or the like, or any combination thereof.

Statement 3. A method according to Statement 1 or 2, where the polymer(s) is/are chosen from organic polymers, biopolymers, and the like, and any combination thereof.

Statement 4. A method according to any one of the preceding Statements, where the impure monomer(s) (which may be recycled monomer(s) or the like) (or impure molecule(s) of interest), structural analog(s) thereof, or the like, or the combination thereof is/are organic acid(s), polyol(s), amine(s) (which may be diamines or the like), any of which may, independently, are protonated or deprotonated, or the like, or any combination thereof.

Statement 5. A method according to any one of the preceding Statements, where the impure monomer(s) (which may be recycled monomer(s) or the like), structural analog(s) thereof, or the combination thereof is/are formed from a textile, an article of manufacture (such as, for example, a container (such as, for example, a bottle, a food trays, a food packaging, or the like), a rug/carpet, a structural element, a surface element (such as, for example, a component or part in an automobile, a plane, or the like), or the like, or any combination thereof.

Statement 6. A method according to any one of the preceding Statements, where the forming the metal complex(es), metal organic framework(s), coordination polymer network(s), or the like, or the combination thereof comprises contacting the impure monomer(s) (which may be recycled monomer(s) or the like) (or impure molecule(s) of interest), structural analog(s), or the combination thereof with one or more metal precursor(s), where the metal complex(es), metal organic framework(s), coordination polymer network(s), or the like, or the combination thereof are formed.

Statement 7. A method according to any one of the preceding Statements, where the decomposing the metal complex(es), metal organic framework(s), coordination polymer network(s), or the like, or the combination thereof comprises contacting (e.g., forming a mixture (which may be a solution or the like) or the like with) the metal complex(es), metal organic framework(s), coordination polymer network(s), or the like, or the combination thereof with one or more acid(s), one or more base(s), one or more organic solvent(s), one or more ionic liquid(s), or the like, or any combination thereof.

Statement 8. A method according to any one of the preceding Statements, where the decomposing the metal complex(es), metal organic framework(s), coordination polymer network(s), or the like, or the combination thereof comprises contacting the metal complex(es), metal organic framework(s), coordination polymer network(s), or the like, or the combination thereof with one or more acid(s) forms metal precursor(s), structural analog(s) thereof, or the like.

Statement 9. A method according to any one of the preceding Statements, where the impure monomer(s) (which may be recycled monomer(s) or the like) (or impure molecule(s) of interest) comprise one or more impurit(ies), structural analog(s) thereof, or the combination thereof and the purified monomer(s) (which may be free monomer(s) or the like) (or purified molecule(s) of interest) and/or structural analog(s) thereof is/are substantially free or free of the impurit(ies) and/or the impure monomer(s) (which may be recycled monomer(s) or the like) (or impure molecule(s) of interest)), structural analog(s) thereof, or the combination thereof comprise one or more additive(s) and the purified monomer(s) (which may be free monomer(s) or the like) (or purified molecule(s) of interest) and/or structural analog(s) thereof is/are substantially free or free of the additive(s).

Statement 10. A method according to any one of the preceding Statements, where the purified monomer(s) (which may be free monomer(s) or the like) (or purified molecule(s) of interest) and/or structural analog(s) thereof is/are substantially colorless or colorless.

Statement 11. A method according to any one of the preceding Statements, further comprising use of the purified monomer(s) (which may be free monomer(s)) and/or structural analog(s) thereof in a polymerization reaction.

Statement 12. A polymer formed from at least a monomer or monomers provided by a method according to any one of them preceding Statements.

Statement 13. A method of forming one or more purified monomer(s) (e.g., purified target monomer(s)) comprising: providing one or more object(s), each object independently comprising: i) one or more polymer(s); and, optionally ii) optionally, one or more impurit(ies), one or more additive(s), or any combination thereof, where the one or more polymer(s) independently comprise a first portion of a first structural unit formed from polymerization reaction of a first target monomer and optionally a second portion of a second structural unit formed from polymerization reaction from a second target monomer; adding first metal precursor (e.g., a first metal-containing material or the like) to form a first metal complex or complexes, a first metal-organic framework (MOF) or MOFs, a first coordination polymer network or network(s), or any combination thereof; optionally, isolating the first metal complex(es), the first metal-organic framework(s) (MOF(s)), and/or the first coordination polymer network(s) or a portion thereof; optionally, decomposing the first metal complex(es), the first metal-organic framework(s), the first coordination polymer network(s), or the combination thereof or a portion thereof, where the first purified target monomer or monomer(s) or a structural analog or analogs thereof is/are formed; and optionally, isolating the first purified target monomer or monomer(s) and/or a structural analog or analogs thereof.

Statement 14. A method according to Statement 13, the method further comprising: adding a second metal precursor (e.g., a second metal-containing material or the like) to form a second metal complex or complexes, a second metal-organic framework (MOF) or MOFs, a second coordination polymer network or networks, or any combination thereof; optionally, isolating the second metal complex(s), the second metal-organic framework(s) (MOF(s)), and/or the second coordination polymer network(s) or a portion thereof; optionally, decomposing the second metal complex(s), the second metal-organic framework, and/or the second coordination polymer network or a portion thereof, where the second purified target monomer, or the second purified target structural analog thereof are formed; and optionally, isolating the second purified target monomer or the second purified target structural analog thereof.

Statement 15. A method according to Statement 13 or 14, the method further comprising heating the one or more object(s) and the first metal precursor (e.g., a first metal-containing material or the like (e.g., to at least 50° C., 100° C., 110° C., 120° C., 130° C., 140° C., 150° C., 160° C., 170° C., 180° C., 190° C., 200° C., or at least 210° C., or in a range from 20° C. and 600° C., including any 0.1° C. value or range therebetween (e.g., from 110° C. to 300° C.) and/or for at least 0.1, 0.5, 1, 2, 3, 4, 5, 6, 7, 8, 9, 10, 11, 12, 13, 14, 15, 16, 17, 18, 19, 20, 21, 22, 23, or 24 hours; or in a range from 5 minutes to 60 minutes or from 1 hour to 72 hours, including any 0.1 minute value or range therebetween (e.g., from 1 hours to 48 hours).

Statement 16. A method according to any one of Statements 13 to 15, where less than 1 wt %, 0.5 wt %, 0.1 wt %, 0.01 wt %, 0.001 wt %, or substantially no base material (e.g. metal hydroxide or the like) or acid is added before forming the first metal complex(s), the first metal-organic framework(s) (MOF(s)), the first coordination polymer network(s), or the combination thereof.

Statement 17. A method according to any one of Statements 13 to 16, where the one or more polymer(s) and the first metal precursor (e.g., a first metal-containing material or the like) form (e.g. directly without a step of forming impure or pre-purified monomers by depolymerization or hydrolysis of the polymer(s)) the first metal complex(s), the first metal-organic framework(s) (MOF(s)), the first coordination polymer network(s), or the combination thereof in one step.

Statement 18. A method according to any one of Statements 13 to 17, where the one or more polymer(s) are depolymerized in the presence of the first metal precursor (e.g., a first metal-containing material or the like) and directly react with the first metal precursor (e.g., a first metal-containing material or the like) to form the first metal complex(s), the first metal-organic framework(s) (MOF(s)), the first coordination polymer network(s), or the combination thereof in one step (e.g., without forming impure target monomers).

Statement 18. A method according to any one of Statements 13 to 17, where the one or more object(s) is/are chosen from a textile, a plastic waste, an article of manufacture, a container, a rug/carpet(s), a structural element, a packaging element, a surface element, and any combination thereof.

The steps of the methods described in the various examples disclosed herein are sufficient to carry out the methods of the present disclosure. Thus, in various examples, a method consists essentially of a combination of the steps of the methods disclosed herein. In various another examples, a method consists of such steps.

The following Examples are presented to illustrate the present disclosure. They are not intended to be limiting in any manner.

### Example 1

This example provides a description of methods and purified monomers of the present disclosure, and uses thereof.

Reactive Crystallization through Metal-Organic-Framework Intermediate Purifies Terephthalic Acid from Textile Impurities. Methods were developed for purifying TPA by crystallization that works in water and does so by forming crystals at the purification temperature (to avoid trapping the impurities at the cooling stage).

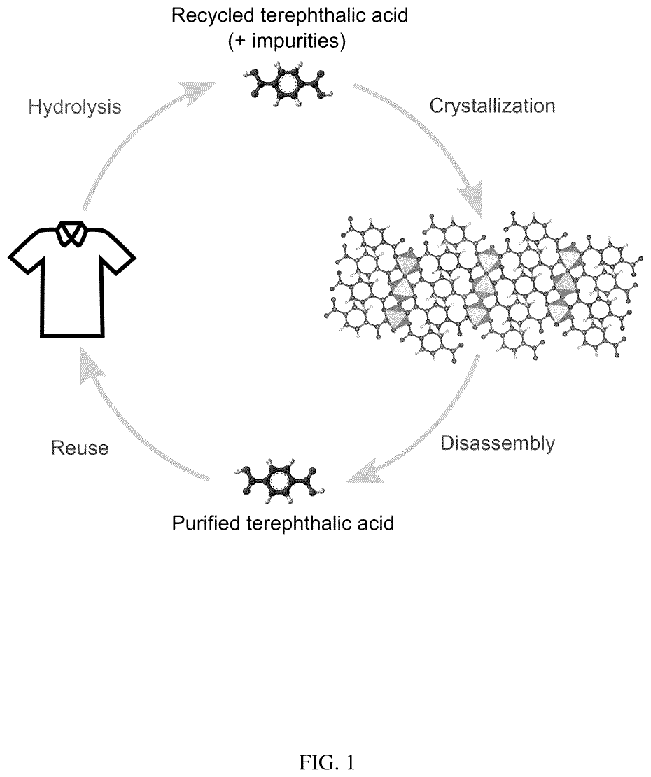

MOFs are compounds consisting of metal ion clusters coordinated to organic ligands to form three-dimensional crystalline structures. TPA derived from PET waste has been used to prepare MOFs, including UiO-66(Zr), MIL-53(Al), and MIL-47(V). In some cases, PET waste can be directly used as the synthesis reagent, including Cr-MOF, MIL-47(V), MIL-53(Cr), MIL-53(Al), MIL-53(Ga), and MIL-101(Cr). This work demonstrated that the MOF disassembly can separate TPA from its impurities. MOFs were selected that can undergo decomposition in strong acid, with the intention to create a final solution resembles the solution at start. It was shown that recycled TPA from blended PET waste can be reversibly assembled as the organic linker of MIL-53(Ga) and its disassembly results in pure TPA. Furthermore, it was shown that the metal salt that can be circularly recycled for the further crystallization (FIG. 1).

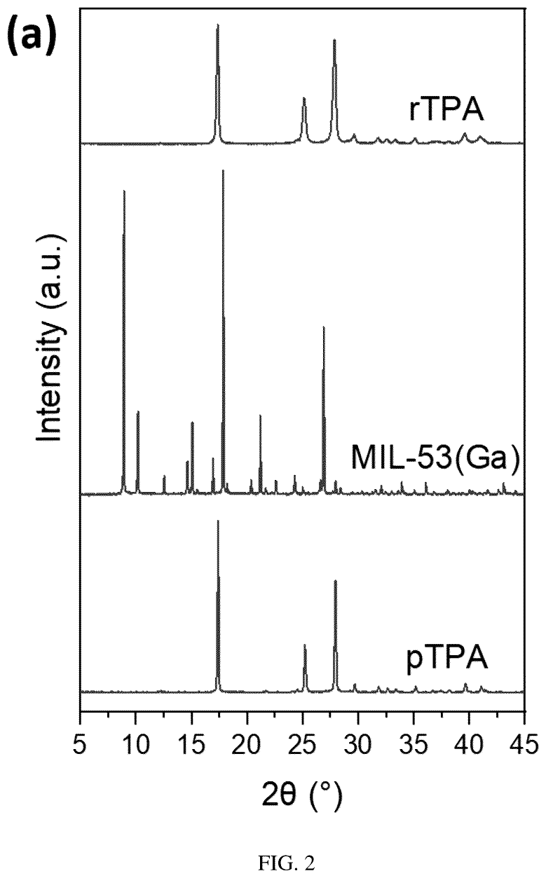

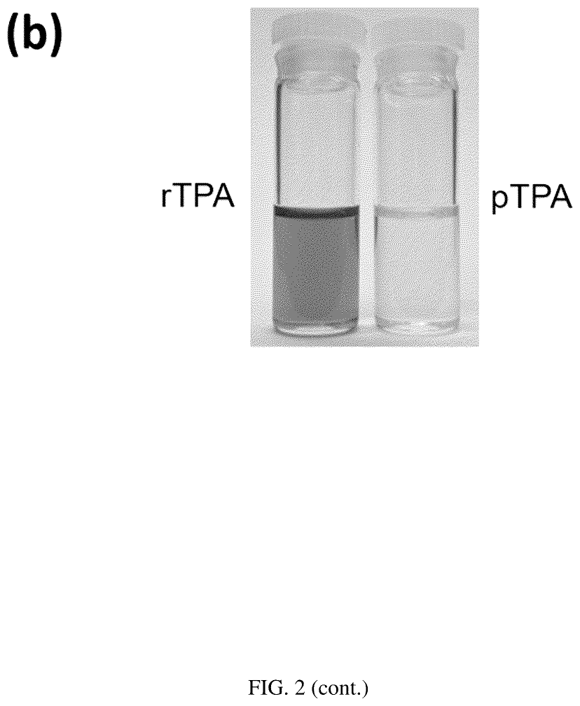

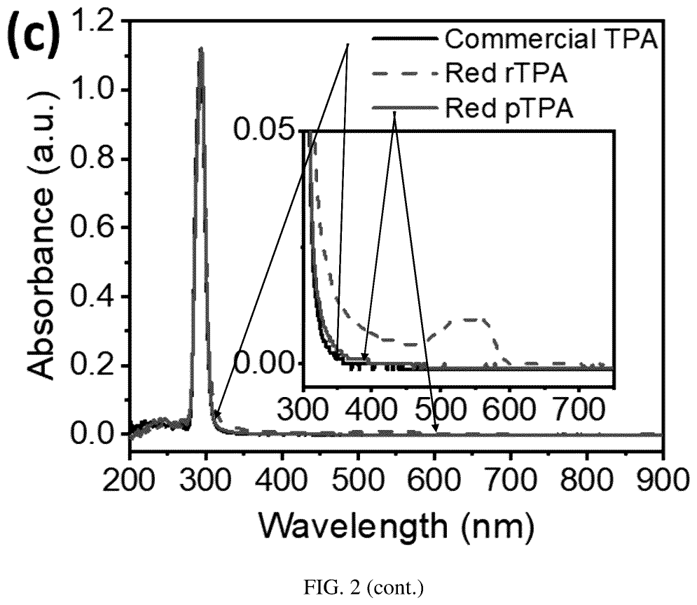

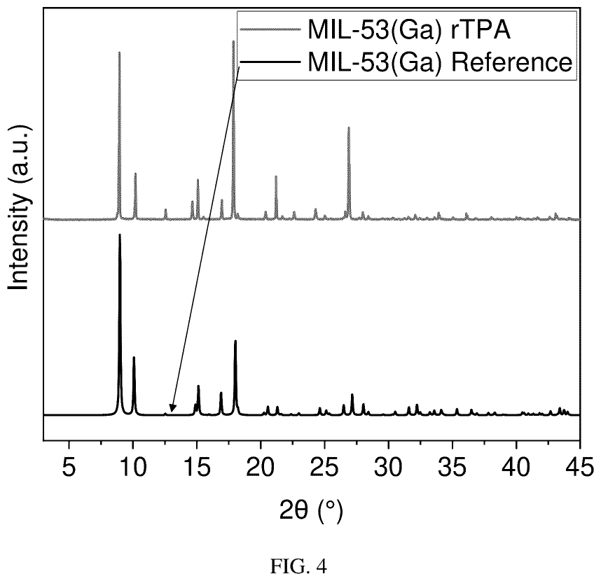

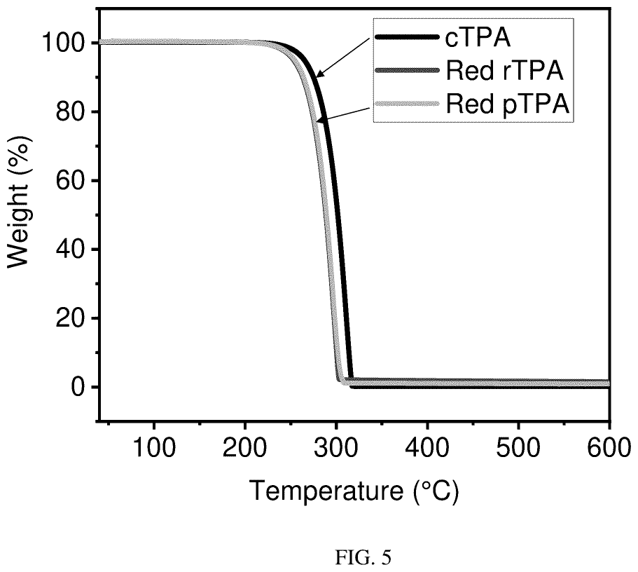

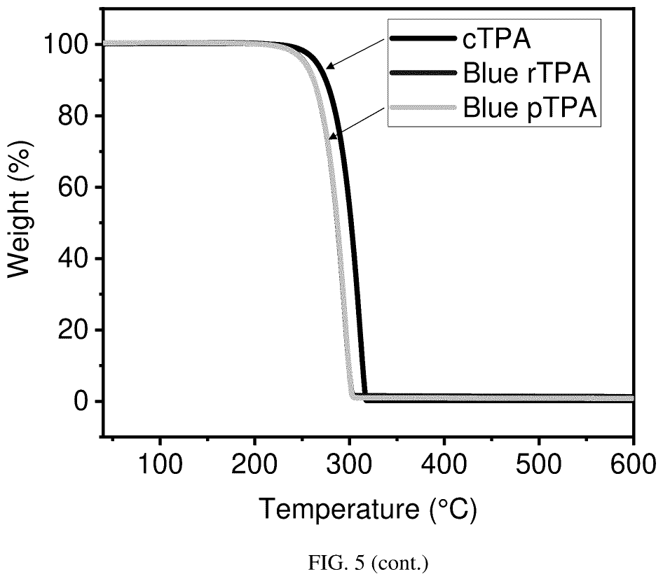

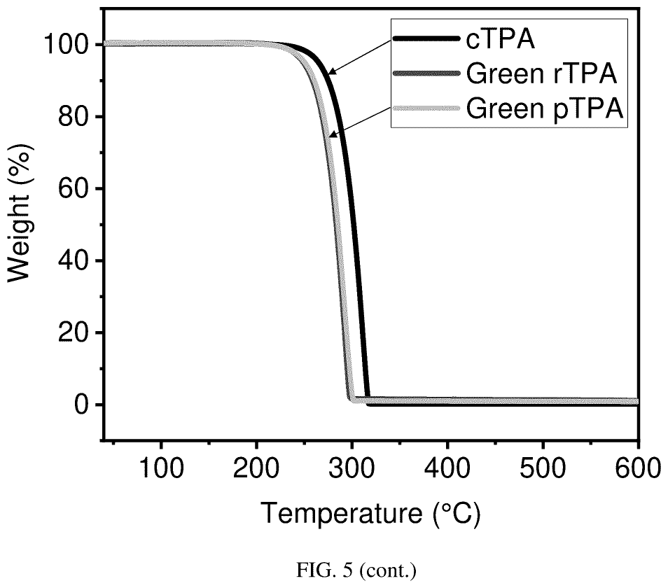

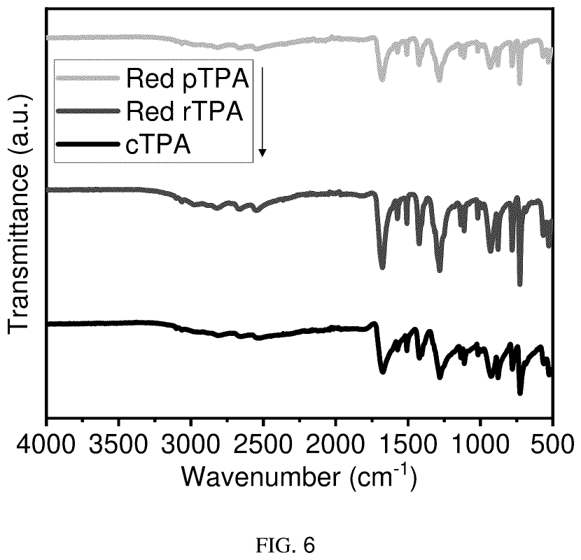

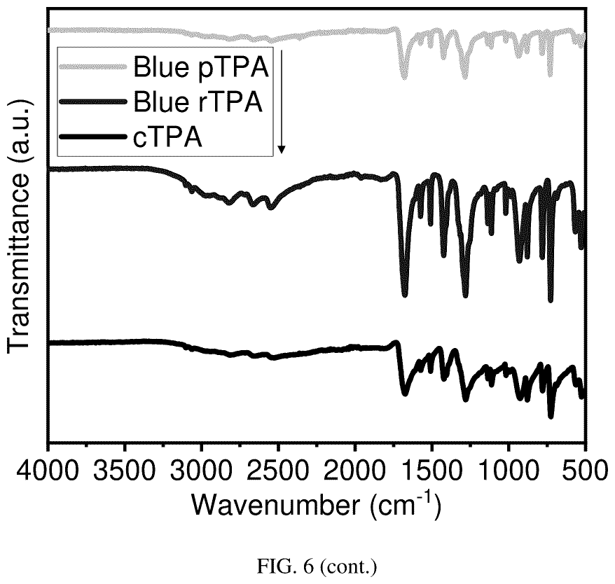

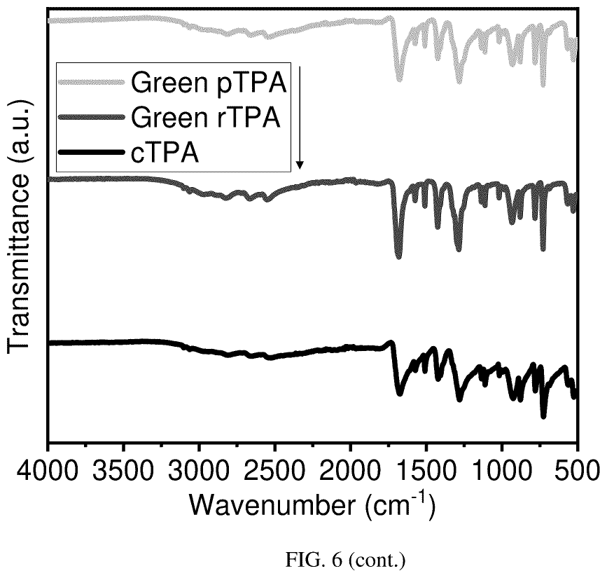

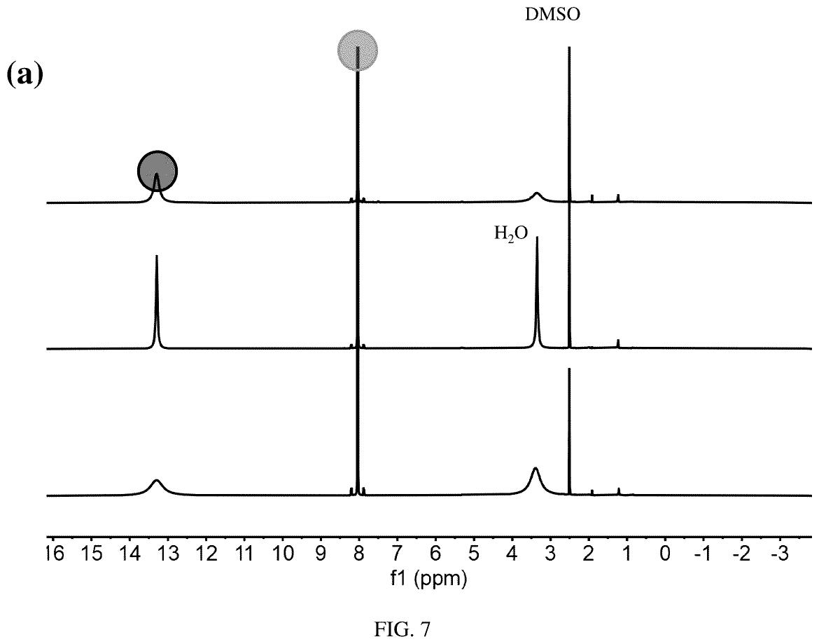

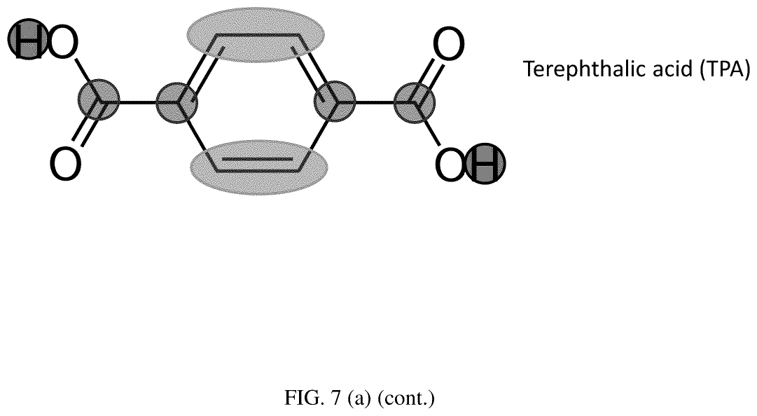

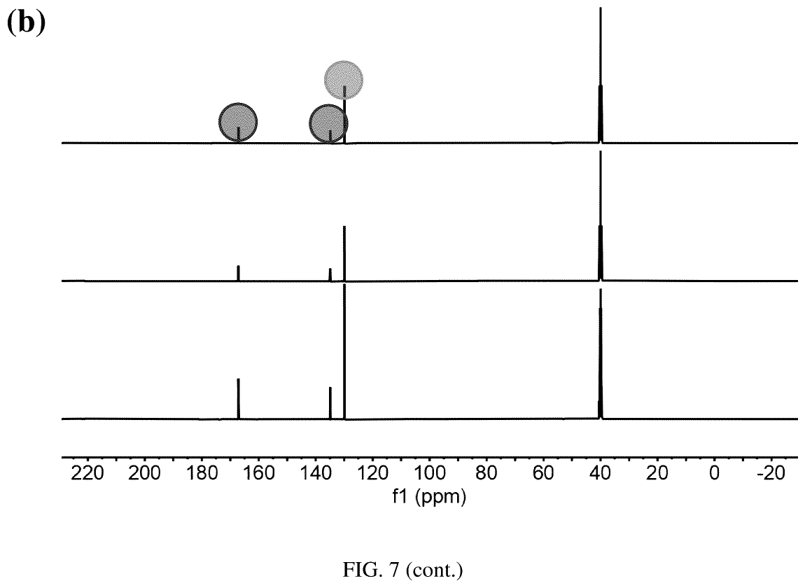

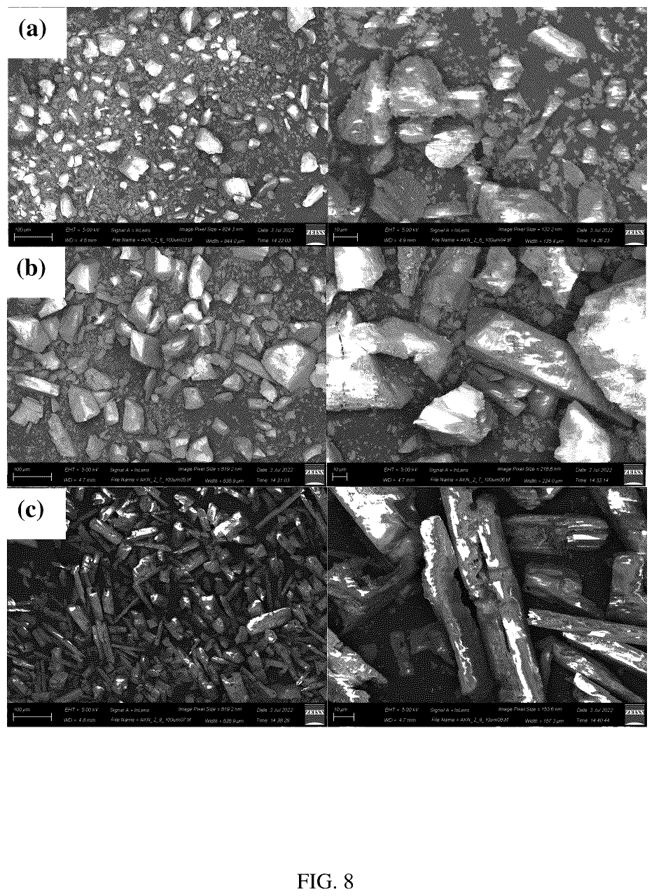

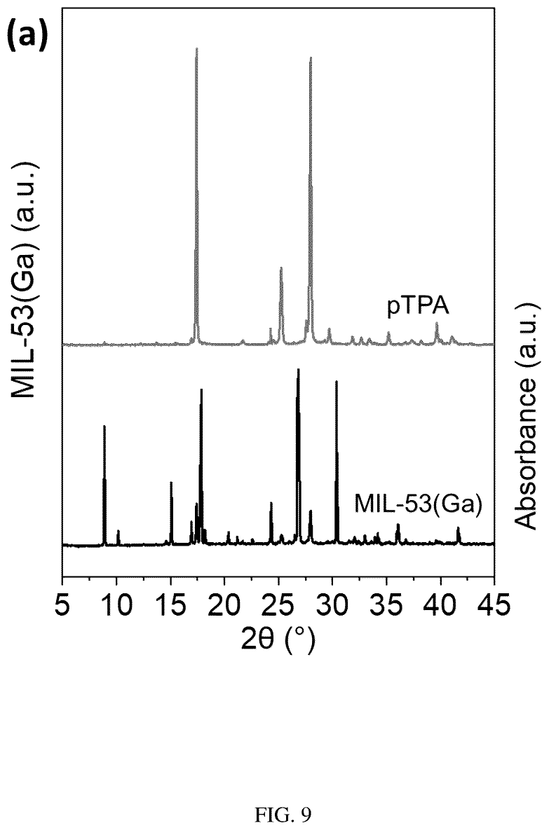

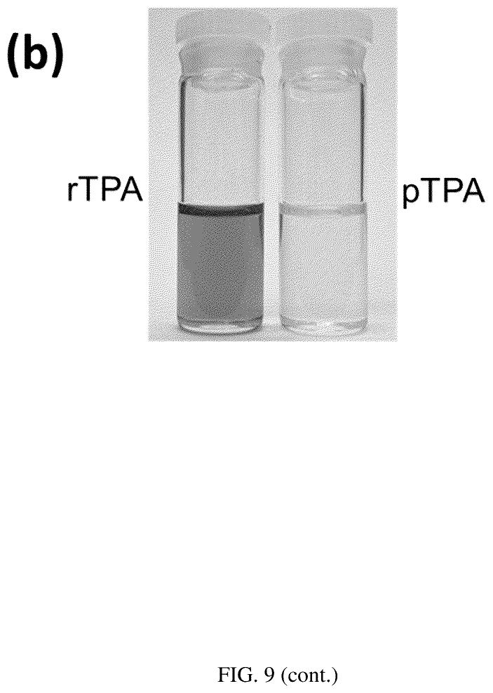

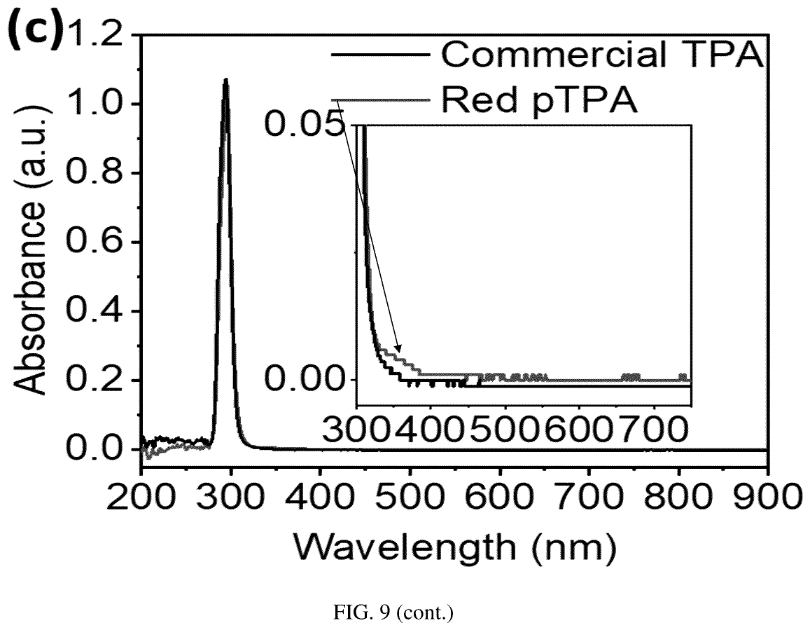

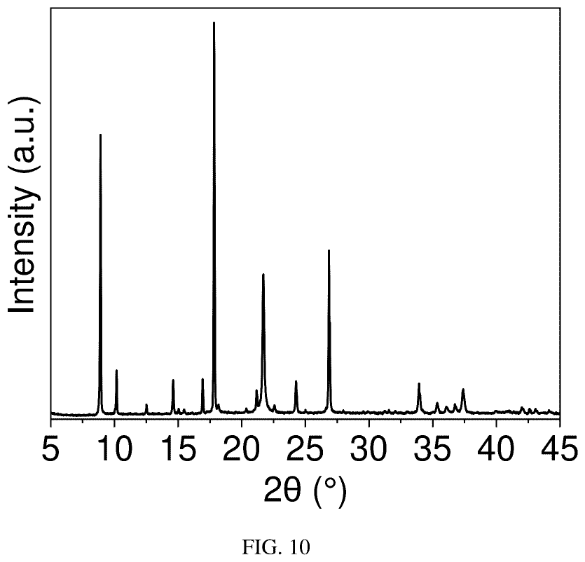

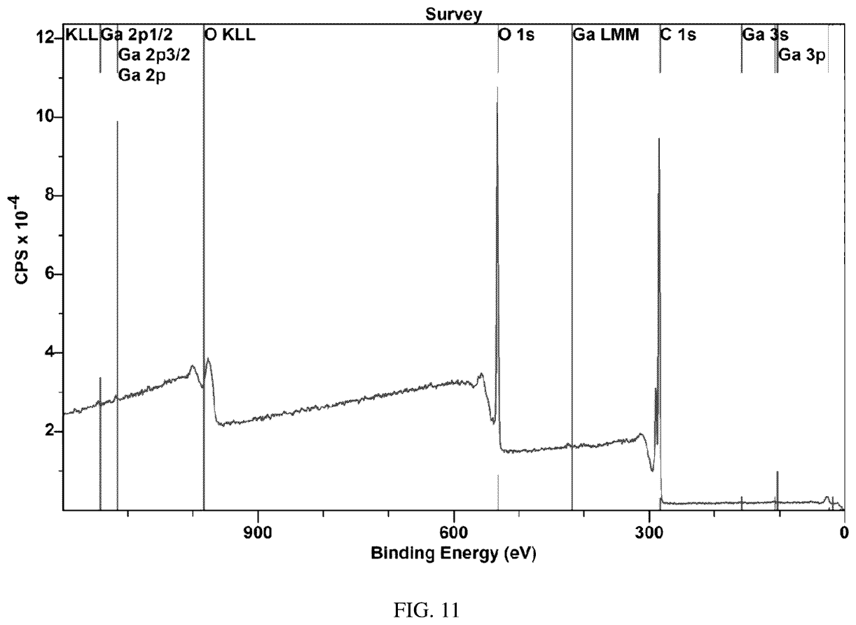

Results and Discussion. Powder X-ray diffraction (PXRD) confirms the formation of rTPA from a red polycotton (65/35 polyester/cotton blend) fabric after alkaline hydrolysis (FIG. 2a and FIG. 4). All observed PXRD peaks are in good agreement with the TPA crystal structure. Repeated water washing, however, did not remove the fabric dyes from rTPA. The leftover impurity was visually recognizable through the color of the TPA and its solution in DMSO (FIG. 2b) but below the detection limit of Nuclear Magnetic Resonance (NMR), Thermogravimetric Analysis (TGA), or Fourier-Transform Infrared Spectroscopy (FTIR) (see FIGS. 5-7). A mixture of rTPA, Ga(NO₃)₃ and water was heated in an autoclave at 210° C. for 5 hours to synthesize MIL-53(Ga) (see FIG. 9-11). The PXRD spectra of the prepared MIL-53(Ga) (FIG. 4) matches well with literature, confirming that rTPA can serve as the organic linker in a metal organic framework synthesis. The particle sizes were on the order of 10-100 μm (see FIG. 8). The MIL-53(Ga) crystals were subjected to boiling HNO₃ to form purified TPA (pTPA). FIG. 2a shows the PXRD of rTPA obtained after the MIL-53 (Ga) disassembly. The peak positions match well the TPA crystal structure and have sharper FWHM than rTPA, indicating the higher degree of crystallinity.

The comparison between rTPA and pTPA dissolved in DMSO shows visual evidence of color change (FIG. 2b). The rTPA solution had a red tint, indicating the presence of dyes and other impurities from the red fabrics, while the pTPA solution was clear and colorless, indicating no dye impurities. Given that the impurity was recognizable through the color of the solution but not detectable by NMR, TGA, or FTIR, the cleanliness of the TPA was quantified using UV-vis spectroscopy after dissolving the solids in DMSO (FIG. 2c.) Comparing the absorbance of rTPA and pTPA to commercial TPA, all three have a peak characteristic of TPA at 300 nm. Commercial TPA has no absorbance other than the characteristic TPA feature at 300 nm. In contrast, rTPA has absorbance peaks that extend as far as 600 nm. These peaks were assigned to the impurities in rTPA, most likely dyes and colorants. The absorbance peaks disappear in pTPA, whose absorbance spectrum matches closely with that of the commercial TPA. This observation suggests that pTPA has purity close to the commercial TPA monomers, validating our hypothesis that reactive crystallization via the MIL-53(Ga) formation rejects textile impurities in recycled monomers.

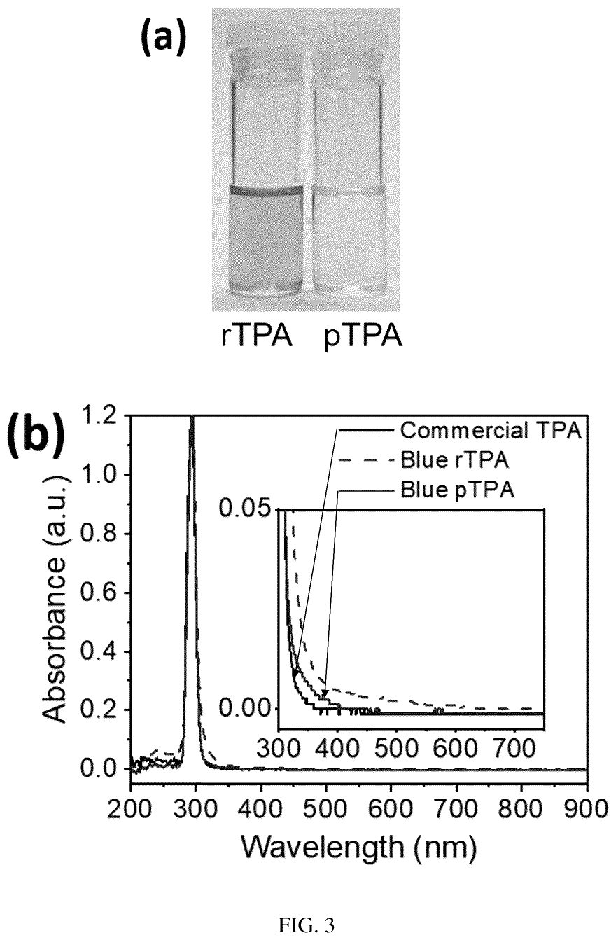

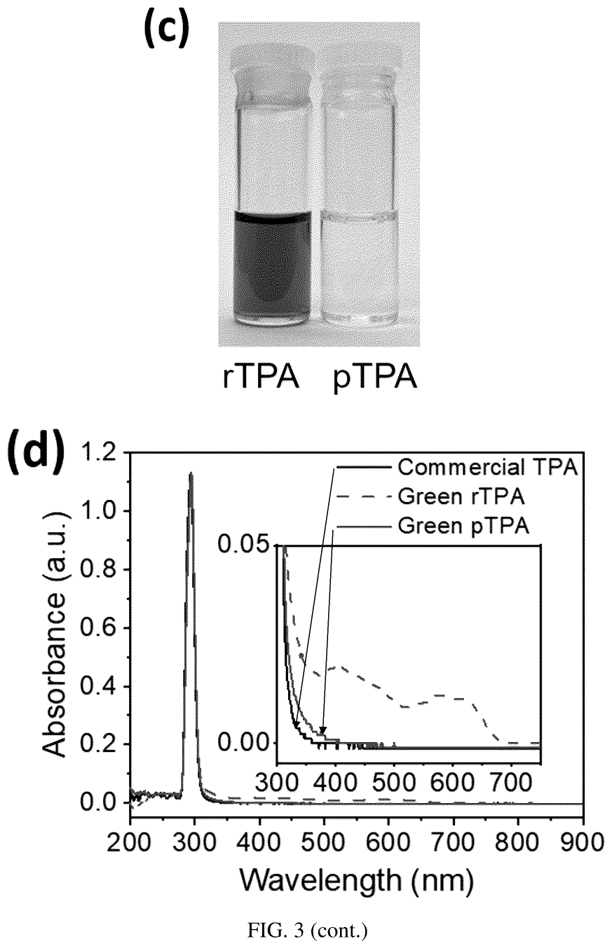

The TPA purification process works irrespective of the color of the starting textile. The reactive crystallization approach was applied to polycotton dyed with different colors (blue: FIG. 3a-b, green: FIG. 3c-d). The photographs of rTPA dissolved in DMSO from the blue and green fabrics showed a tinted solution, indicating the presence of impurities, similar to the red polycotton sample. This effect was quantified using UV-Vis spectroscopy. FIG. 3b,d compares the spectra of rTPA, pTPA, and commercial TPA, where pTPA went through the same methodology as red polycottons. The results showed the same outcome: the rTPA showed only the 300 nm peak that is the characteristic absorption of TPA, and no other absorbance features. It is noteworthy that the base absorbance of the rTPA was slightly higher than the commercial TPA, although it was difficult to quantify given that it presented as a very weak signal.

In summary, the purification of recycled TPA from waste polyester textiles through reactive crystallization was demonstrated. The instant approach crystallizes recycled TPA with Ga nitrate to form a MIL-53(Ga) MOF intermediate, which was subsequently disassembled in nitric acid to extract purified TPA. The reported process is water-based and does not require organic solvents. Colorimetric analysis using DMSO as a solubilizing solvent showed that purified TPA produced a clear solution. This observation is in contrast to the tint pre-purified TPA solution, which contained dyes from the depolymerization step. This result confirmed the removal of impurities such as textile colorants. The processs produced in addition to purified TPA, Ga nitrate and excess nitric acid that can be reused in the future reactive crystallization step. This work demonstrated an aqueous method for purifying TPA from impurities and contribute a technique for purifying recycled waste textiles to enable circular polyesters.

Experimental Details. Reagents and Chemicals. Gallium (III) nitrate hydrate (Ga(NO₃)₃·xH₂O, Beantown Chemical, 99.9%), terephthalic acid (BDC, Sigma Aldrich, 98%), sodium hydroxide (NaOH, Macron Fine Chemicals), polyester/cotton shirts (Port & Company, 65% polyester 35% cotton), acetone (Sigma-Aldrich, 99.9%), methanol (Sigma-Aldrich, 99.8%), sulfuric acid (Honeywell, 95.0-98.0%), and nitric acid (LabChem, 50% v/v) were used as-received. All water used was deionized (MilliporeSigma, Direct-Q 5UV-R).

Depolymerization of PET Textile Wastes. The polyester cotton fabric was depolymerized following an alkaline hydrolysis procedure from Palme et al. 20 g NaOH and 480 g deionized water were combined in a 500 mL round bottom flask to make a 10 wt % NaOH alkaline hydrolysis solution. 5 g of either red, green, or blue 65/35 polyester/cotton fabric was cut into 1 in x 1 in pieces and put into the reaction flask. The solution was heated to 100 C and stirred for 200 min (min=minute(s)). The round bottom flask was cooled to room temperature. The liquid fraction was extracted using a vacuum filtration using a glass microfiber filter. To the liquid phase sulfuric acid was added to precipitate TPA solids. TPA was filtered off using vacuum filtration with a glass microfiber filter and washed with deionized water. Prior to PXRD, TPA was dried at 150° C. overnight.

Synthesis of MIL-53(Ga). MIL-53 (Ga) was hydrothermally synthesized under autogenous pressure from a mixture of 0.525 g Ga nitrate, 0.38 g terephthalic acid, and 10 g deionized water. The reactants were mixed together in a 100 mL Teflon-lined autoclave, which was heated at 210 C for 5 hr. After the reaction, the product was washed with N,N-dimethylformamide (DMF) to remove excess TPA and acetone to help the product dry more quickly. Without using DMF, PXRD showed that the resulting product contained excess TPA.

Disassembly of MIL-53(Ga). MIL-53(Ga) was disassembled to purified TPA by stirring the MOF with the 2 mole-equivalent amount of nitric acid. The mixture was stirred at 100° C. for 6 hr in a lidded glass vial with a needle vent. After cooling to room temperature, the mixture was centrifuged 3 times with water to remove remaining nitric acid and once with MeOH to help dry the product more quickly. The product was then dried and prepared for characterization.

Characterization. The Bruker D8 Advance ECO powder diffractometer with a 1 kW Cu-Kα source was used to collect PXRD in the range of 2θ=3-50° at a scanning rate of 0.02° s₋₁. All samples were dried overnight. UV-Vis spectra was recorded on UV-Vis spectrometer (Shimadzu) in the range of 200-900 nm. Blank DMSO as a background for all UV-Vis experiments.

### Example 2

This example provides a description of methods and purified monomers of the present disclosure, and uses thereof.

The extraction of high-purity benzene-1,4-dicarboxylic acid (BDC), the major PET monomer from textiles, is, therefore, a major technical hurdle in textile circularity. The extraction of high-quality BDC from PET fibers in textiles was demonstrated. The approach uses reactive crystallization to turn PET directly into a metal-organic framework (MOF) using only metal salts and water as chemical inputs. The process is base-free and organic-solvent-free. As BDC is the only component in this mixture capable of forming an extended, crystalline MOF network, the BDC monomers are separated from impurities as the MOF crystallizes. This concept was demonstrated on a post-consumer PET fabric, extracting colorless BDC monomers spectroscopically reminiscent of a virgin-grade material as the final product. Systematic control of the reaction parameters reveals the MOF assembly mechanism and the importance of the reaction conditions in promoting the metal-BDC complexation step prior to the MOF assembly.

This EXAMPLE describes a strategy for integrating the depolymerization and purification steps into one using metal-organic frameworks (MOFs). It was demonstrated that this approach can turn colored PET textiles into clean, colorless monomers spectroscopically reminiscent of petrochemically derived materials. The strategy takes advantage of the common usage of BDC as a constituent in MOFs, a class of porous crystalline solids composed of metal-based secondary building units coordinated to multitopic organic ligands in a two- or three-dimensional ordered framework. It was shown (as described in EXAMPLE 1) that BDC isolated from the alkaline hydrolysis of PET textiles can be purified using Ga³⁺ as the coordinating metal ion. This entirely aqueous-based process can return colorless BDC, reminiscent of virgin-grade materials. Nonetheless, the method requires several additional steps to facilitate the purification, including pretreatment of the polyester fabric with sodium hydroxide and sulfuric acid to recover the organic linker, BDC.

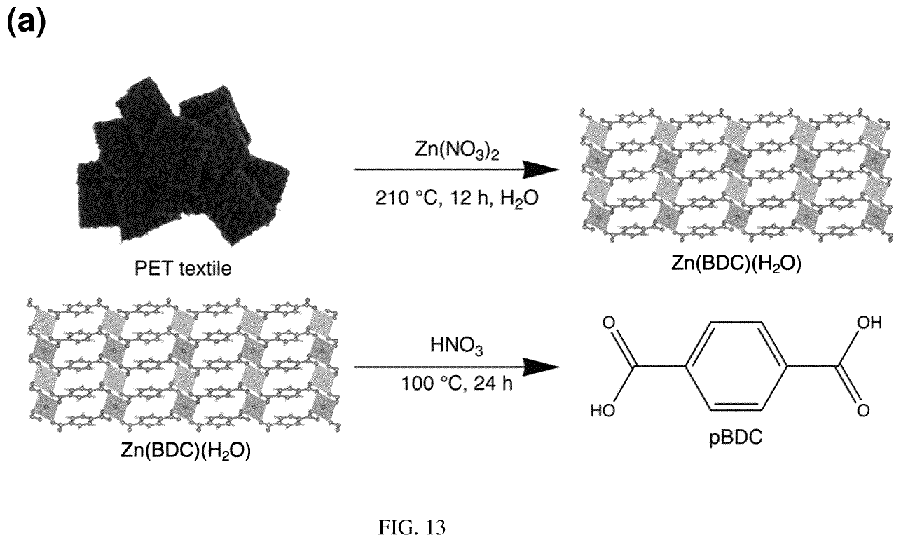

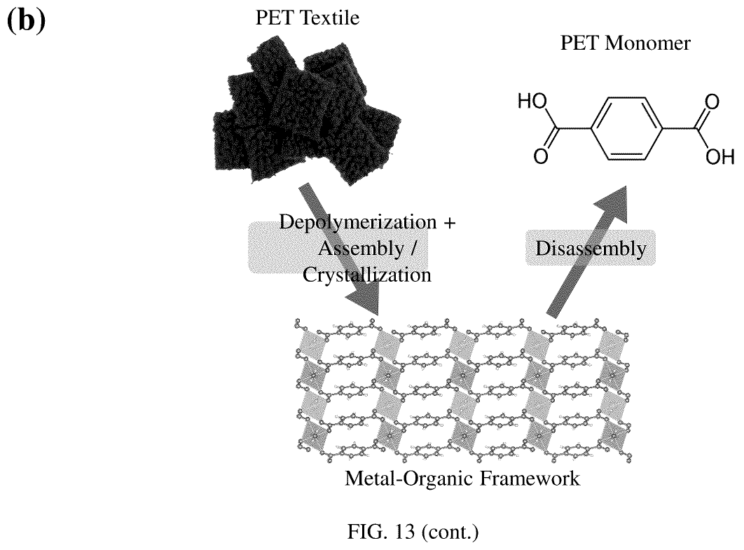

Driven to simplify the steps involved in recycling, an integrated process that combines depolymerization and reactive crystallization into one procedure was developed. There was motivation to minimize the number of steps and remove the need to use base and acid as the depolymerization catalyst and pH adjuster, respectively. Valh et al. showed that PET textiles can be depolymerized directly under hydrothermal conditions; however, the base-acid purification steps were still needed to achieve a purity suitable for repolymerization. The instant process eliminates the need for a caustic, one of the cost factors in hydrolysis-based PET recycling, by directly transforming PET plastics into MOFs, followed by a subsequent disassembly to monomers. MOFs were previously directly synthesized from PET bottles, such as Cr-MOF, MIL-47(V), MIL-53(Cr), MIL-53(Al), MIL-53(Ga), and MIL-101(Cr) (MIL=Matériaux de l'Institut Lavoisier). However, unlike prior works, the instant development was constrained to aqueous processing to avoid using organic solvents. Also, there was focus on PET textiles instead of bottles, as the former contains significantly more impurities. It was shown that an extended network of Zn and terephthalate can be directly assembled from PET and disassembled into pure BDC monomers (FIG. 13).

The instant approach starts by identifying reaction conditions that enable aqueous depolymerization and finding MOFs that can be formed in the same environment. There was ocus on metastable MOFs, with the rationale that their crystallization can be kinetically controlled but must not be too thermodynamically favorable to inhibit subsequent disassembly. The first step was to select metal ions that can withstand the depolymerization condition and coordinate with BDC to assemble a metastable network. The metastable structures of the Zn-BDC MOF family were chosen. In this MOF family, synthesis conditions (e.g., time, pressure, temperature, and concentration) have been reported to drastically affect the obtained crystal structures and morphologies, even from the same reactants, revealing multiple competing forces in the self-assembly kinetics. Researchers have used this phenomenon to create different metastable Zn-BDC MOFs by varying the reaction variables between Zn salts, BDC, and H₂O. The Zn-BDC MOF metastability was leveraged by transforming PET into Zn MOF intermediates, followed by a subsequent disassembly that turns Zn MOFs into the final purified BDC monomers.

General Procedures. For each sample, Pawley refinements were performed in TOPAS V6 on all distinct phases identified by PXRD, including those solved through indexing, to obtain precise unit cell parameters using a double-Voight method. For samples containing multiple phases, figures of merit reported for their refinements describe the accuracy of the fits when refining on all identified phases simultaneously. Reflections in the PXRD pattern of 1-1-Zn-BDC-72 h (h=hour(s)) corresponding to the unknown phase Unknown 1 were identified by visual inspection (30 total) and indexed using TOPAS V6. From the solutions, a possible unit cell of Unknown 1 was identified: space group=Cc, a=5.737 Å, b=8.9161 Å, c=16.0251 Å, α=γ=90°, β=87.742°, GoF=6.71. While this is a likely solution based on the current data, it is acknowledged that other indexing solutions matched the selected peak positions as well. Without further structural information these unit cell parameters are presented as probable but not definitive unit cell parameters of Unknown 1. CCDCs used: BDC: CCDC—1269122; Zn(BDC)(H₂O): CCDC—171952; Zn(BDC)(H₂O)₂: CCDC—171953; ECATIO:CCDC—1520407.

Experimental Methods. Reagents and Chemicals. Red polyester twill (100% polyester, pre-consumer) was purchased from Fabric Wholesale Direct. A post-consumer black polyester fleece was purchased from a secondhand clothing store (100% PET, as labeled by the tag). Both polyester textiles are assigned as 100% PET for mass-balance calculations and were used as received. Zinc nitrate hexahydrate (Zn(NO₃)₂·6H₂O, Sigma Aldrich, 98%), gallium (III) nitrate hydrate (Ga(NO₃)₃·xH₂O, Beantown Chemical, 99.9%), aluminum nitrate nonahydrate (Al(NO₃)₃·9H₂O, Sigma Aldrich, >98%), aluminum chloride hexahydrate (AlCl₃·6H₂O, Sigma Aldrich, 99.9%), benzene-1,4-dicarboxylic acid (BDC, Sigma Aldrich, 98%), acetone (Sigma-Aldrich, 99.9%), nitric acid (LabChem, 50% v/v), hydrochloric acid (HCl, Sigma Aldrich, 37%), ethylene glycol (Sigma Aldrich, 99.8%) and dimethyl sulfoxide (DMSO, Fisher chemical) were used as received. Deionized water was obtained from a water purification system (Millipore-Sigma, Direct-Q 5UV-R.)

Synthesis of MIL-53(Ga). An attempt to synthesize MIL-53(Ga) hydrothermally under autogenous pressure from PET fabric and BDC was carried out. For MIL-53(Ga) synthesis using PET, PET fabric (1 mmol, 0.192 g), Ga(NO₃)₃·xH₂O (1.07 mmol, 0.274 g), and 10 mL of deionized water were mixed in a 100 mL Teflon-lined autoclave, which was heated at 210° C. for 12 h. For MIL-53(Ga) synthesis using BDC, BDC (2.287 mmol, 0.38 g), Ga(NO₃)₃·xH₂O (1.917 mmol, 0.525 g), EG (2.287 mmol, 128 μL), and 10 mL of deionized water were mixed in a 100 mL Teflon-lined autoclave, which was heated at 210° C. for 5 h. This procedure was adapted from previously known work. The reaction mixtures were allowed to cool to room temperature and the solids were isolated via centrifugation. The resulting solid was rinsed with 40 mL deionized water and 10 mL acetone.

Synthesis of MIL-53(Al). The procedure was adapted from Loiseau et al. MIL-53(Al)—NO₃ and MTL-53(Al)—Cl were hydrothermally synthesized under autogenous pressure separately using Al(NO₃)₃·9H₂O and AlCl₃·6H₂O, respectively. PET fabric (5.188 mmol, 0.997 g), Al(NO₃)₃·9H₂O (5.188 mmol, 1.957 g) or AlCl₃·6H₂O (5.188 mmol, 1.253 g), and 30 mL of deionized water were mixed in a 100 mL Teflon-lined autoclave, which was heated to 210° C. for 12 h. The reaction mixture was allowed to cool to room temperature and the solids were isolated via centrifugation. The resulting solid was rinsed with 40 mL deionized water and 10 mL acetone. Then, the products were dried on a hotplate at 100° C. overnight before characterization.

Synthesis of Zn-MOF Materials. A typical Zn-MOF synthesis was performed as follows. Zn-MOF was hydrothermally synthesized under autogenous pressure from a mixture of PET fabric (5.19 mmol, 0.997 g), Zn(NO₃)₂·6H₂O (5.19 mmol, 0.983 g), and 30 mL of deionized water. The procedure was adapted from Yang et al. The reactants were mixed in a 100 mL Teflon-lined autoclave before heating up to 210° C. for 12 h. The reaction mixture was allowed to cool to room temperature and the solids were isolated via centrifugation. The resulting solid was rinsed with 40 mL deionized water and 10 mL acetone. Then, the products were dried on a hotplate at 100° C. overnight before characterization.

Synthesis of hydrothermal BDC (hBDC). hBDC was hydrothermally synthesized under autogenous pressure from a mixture of PET fabric (5.19 mmol, 0.997 g) and 30 mL of deionized water. The reactants were mixed in a 100 mL Teflon-lined autoclave before heating up to 210° C. for 12 h. The reaction mixture was allowed to cool to room temperature and the solids were isolated via centrifugation. The resulting solid was rinsed with 40 mL deionized water and 10 mL acetone. Then, the products were dried on a hotplate at 100° C. overnight before characterization.

Decomposition of MIL-53(Al). MIL-53(Al) was disassembled by stirring the MOF with 7 molar equivalents of concentrated nitric acid. The mixture was stirred at 100° C. for 24 h in a lidded glass vial. After cooling the mixture to room temperature, the mixture was centrifuged five times with 10 mL deionized water per run to remove the remaining nitric acid and then once with 10 mL acetone. The product was dried on a hotplate at 100° C. overnight before characterization.

Decomposition of Zn-MOF. Zn-MOF was disassembled by stirring the MOF with 30 molar equivalents of concentrated nitric acid. The mixture was stirred at 100° C. for 24 h in a lidded glass vial. After cooling the mixture to room temperature, the mixture was centrifuged five times with 10 mL deionized water per run to remove the remaining nitric acid and then once with 10 mL acetone. The product was dried on a hotplate at 100° C. overnight before characterization. The yield of pBDC was 93%±2.84.

Characterization. The powder diffractometer (Bruker D8 Advance ECO) with a 1 kW Cu Kα source was used to collect powder X-ray diffraction (PXRD) patterns at a scanning rate of 0.02° s⁻¹. All samples were dried overnight and ground with mortar and pestle before PXRD characterization. SEM images were collected on a Zeiss Gemini 500 scanning electron microscope. The samples were Au/Pd sputtered before imaging. The SEM was operated at an acceleration voltage of 3 kV with a working distance of 5.1 mm. UV-vis spectra (Shimadzu UV-2600i UV-vis spectrophotometer) were recorded in the 200-900 nm range. Blank DMSO was used as a background for all UV—vis experiments. UV—vis samples were run at 1 mM concentration in DMSO. Here, 0.1 mmol of each BDC sample was added to 10 mL DMSO and then diluted to 1 mM. XPS (Scienta Omicron ESCA-2SR) were analyzed at 10-9 Torr pressure. Monochromatic Al Kα X-rays (1486.6 eV) with photoelectrons were collected from a 1.1 mm diameter analysis spot. Photoelectrons were collected at a 900 emission angle and a 54.7° source-to-analyzer angle. A hemispherical analyzer determined electron kinetic energy, using a pass energy of 200 eV for wide/survey scans and 50 eV for high-resolution scans. A flood gun was used for charge neutralization of nonconductive samples. Colorimetric analysis was carried out by dissolving 5 mg of each BDC sample in glass vials of 2 mL DMSO. A change in the color of the solution was noted by observation. Thermogravimetric decomposition profiles were collected on a Q500 V6.7 thermogravimetric analyzer (TGA) using a temperature ramp of 3.00° C./min from room temperature to 600.00° C. under an atmosphere of zero-grade air (20-22% O₂ in N₂). Data analysis was performed using the TRIOS software package. Attenuated total reflectance infrared (ATR-IR) spectra were collected on a Bruker Tensor II spectrometer equipped with a diamond ATR attachment. ¹H and ¹³C NMR data were collected on a Bruker INOVA 500 MHZ spectrometer in DMSO-d₆ solvent.

Results and Discussion. A series of metal ions were surveyed to determine which could directly turn PET into a MOF via reactive crystallization without needing to hydrolyze PET beforehand. Ga³⁺, which was used in previous work (e.g., described in EXAMPLE 1), was initially used to transform impure BDC into MIL-53(Ga) MOF. Unfortunately, no MIL-53(Ga) formation was observed when combining the PET with Ga(NO₃)₃·xH₂O. Irrespective of the hydrothermal conditions, only BDC was observed (based on PXRD analysis), which suggested that PET hydrolysis occurred but not MOF crystallization. Given that MIL-53(Ga) assembly was observed when subjecting recovered BDC to Ga(NO₃)₃·xH₂O, it was hypothesized that the presence of impurities in the sample hampered the MIL-53(Ga) formation. To validate that this observation is not due to the presence of ethylene glycol, the other monomer produced upon PET hydrolysis, MIL-53(Ga) was synthesized using BDC, Ga(NO₃)₃·xH₂O, and ethylene glycol, following a previously known literature procedure. This experiment produced MIL-53(Ga), as supported by PXRD, suggesting that ethylene glycol is likely not the factor limiting MOF formation.

Al³⁺ was next tested due to the well-known hydrothermal synthesis of MIL-53(Al). When treating PET with either Al(NO₃)₃·9H₂O or AlCl₃·6H₂O under hydrothermal conditions, the successful formation of crystalline MIL-53(Al)—NO₃ and MIL-53(Al)—Cl, respectively, was observed. However, no condition to disassemble the obtained MIL-53(Al) back to BDC using acid was found. It was hypothesized that, based on the hard and soft acid and base theory, high valence Al³⁺ (a hard Lewis acid) binds strongly to the carboxylate linker BDC²⁻ (a hard Lewis base), making MIL-53(Al) highly stable. Using excessive temperature and time causes the reaction to form insoluble precipitates, such as AlOOH (boehmite), as shown by the post-drying PXRD.

It became evident that the ideal coordinating metal should not bind to BDC so strongly that disassembly requires harsh conditions and inevitable side reactions, as was the case with Al³⁺. It was hypothesized that Zn²⁺, a lower valence and borderline hard/soft Lewis acid known for its ability to coordinate with BDC to form water-sensitive MOFs such as MOF-5 and MOF-2, would work well for this purpose. Several papers have shown that these MOFs transform into different crystalline phases when exposed to water. For example, MOF-5 (Zn₄O(BDC)₃) undergoes phase transitions at water concentrations higher than 0.6 mol/L. Similarly, MOF-2 ([Zn(BDC)(H₂O)]·DMF) undergoes a structural transition to a different crystalline zinc phase, Zn(BDC)(H₂O)₂, when it is humidified. The extreme sensitivity of Zn-MOFs makes the solvent selection, whether aqueous or non-aqueous, the choice of the anions, and other crystallization conditions essential in controlling the interaction between Zn²⁺ and BDC. It was hypothesized that synthesizing a Zn-BDC MOF using water instead of an organic solvent could directly make these crystalline phases, allowing for a greener synthesis route. Another advantage of Zn²⁺ over most other metal cations is its relatively low cost and high abundance.

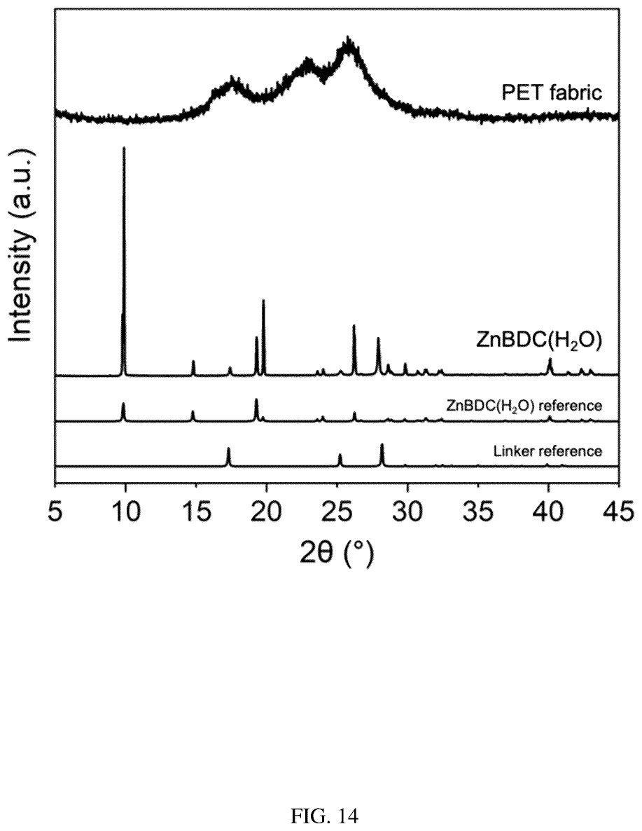

To synthesize Zn-MOF from PET, a mixture of red PET fabric, Zn(NO₃)₂·6H₂O, and water was heated in a Teflon-lined autoclave at 210° C. for 12 h. The PXRD pattern of the prepared Zn(BDC)(H₂O) MOF (FIG. 14) matches well with previous literature, confirming that impure PET fabric can serve as the organic linker source in the synthesis. The resulting MOF contained BDC linkers, as confirmed by PXRD.

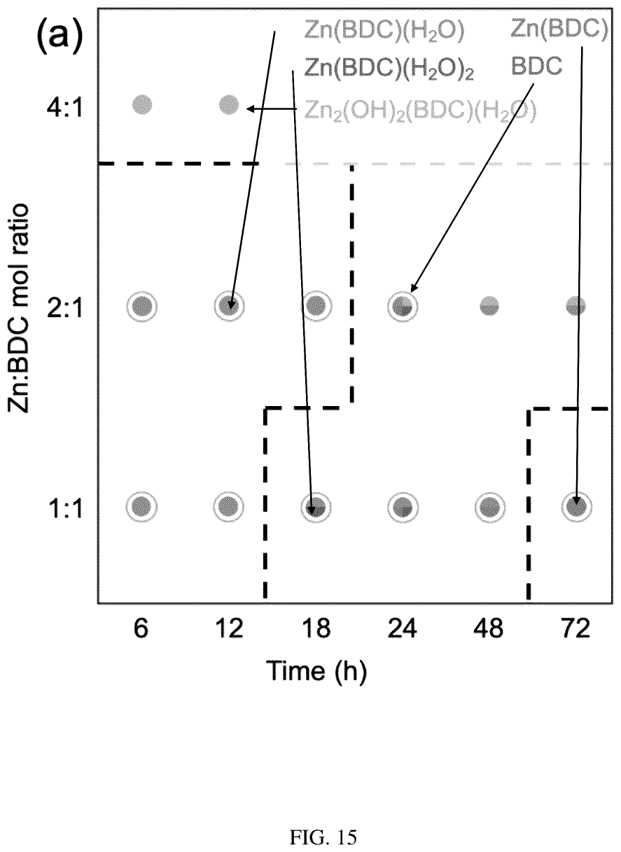

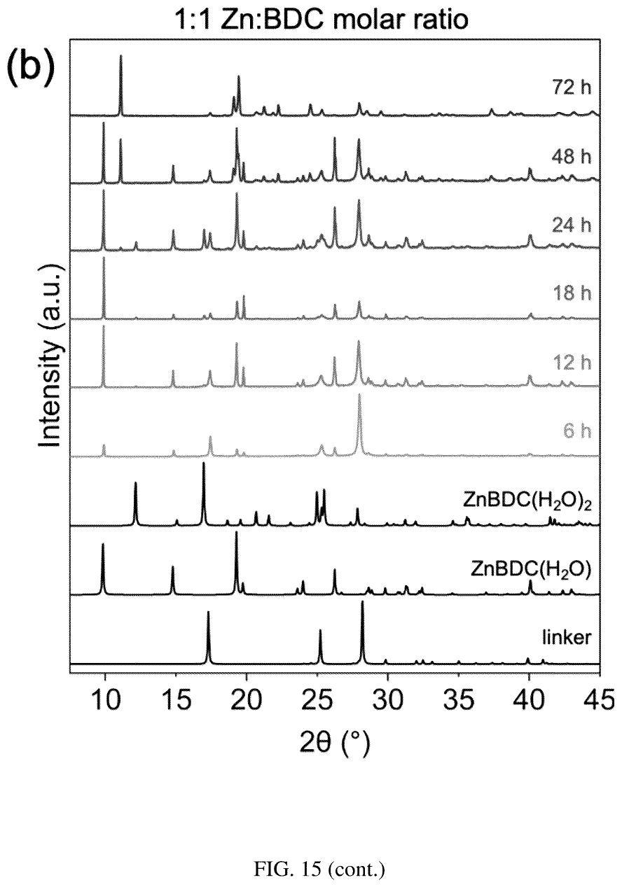

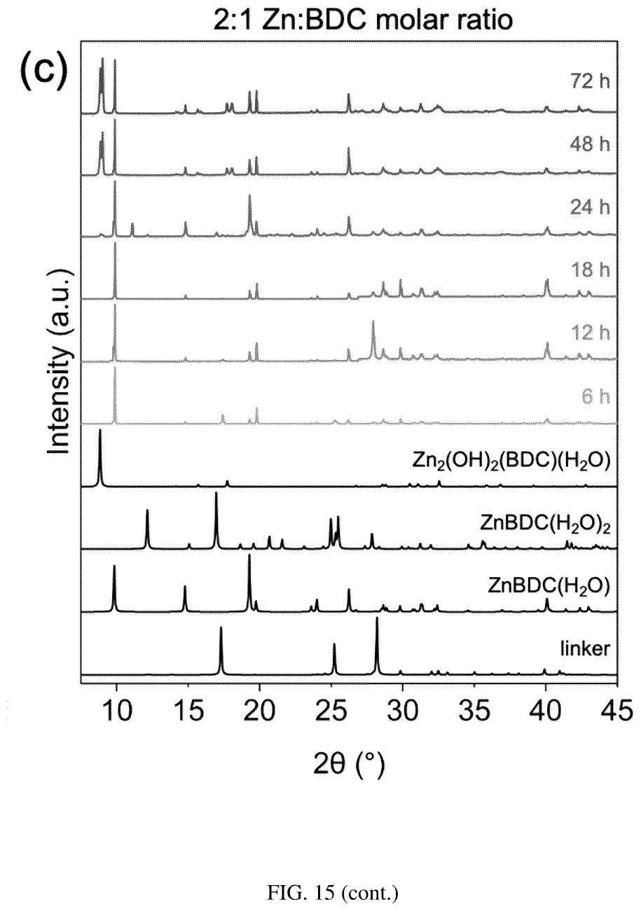

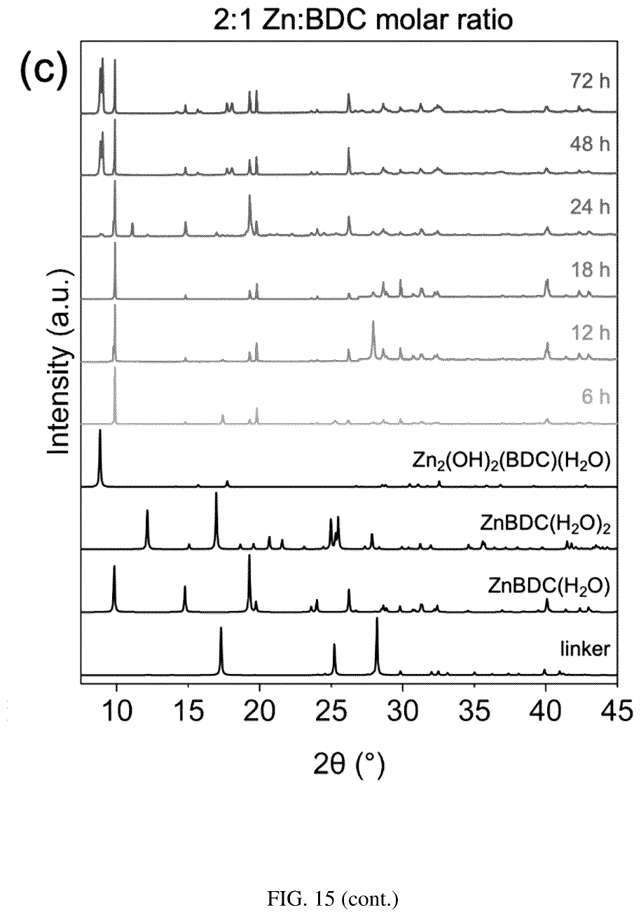

Various molar ratios of Zn:BDC and reaction times were tested to determine the effect of synthesis conditions on MOF formation (FIG. 15). Previous studies have shown that Zn-MOFs can be synthesized with water as a solvent using a 1:1:2 molar mixture of Zn:BDC:NaOH in a hydrothermal reaction vessel. The instant procedure was adapted from these conditions but omitted the NaOH to eliminate the use of a caustic base, a drawback of most PET hydrolysis systems. A mixture of Zn(NO₃)₂·6H₂O, PET textile, and water in a mol ratio of 1:1:320 was reacted hydrothermally. Through PXRD analysis and Pawley refinement of the data, successful depolymerization and partial assembly of PET into a crystalline network was observed at reaction times of 6 hours and greater (see FIG. 15(b)). After 6 hours, new reflections are observed with the most intense peaks at 2θ=9.8°, 14.7°, and 19.8°, matching Zn(BDC)(H₂O). At 18 hours, there is growth of an intermediate structure Zn(BDC)(H₂O)₂ with the most intense peaks at 2θ=12.2° and 16.9°. At 24 hours, both Zn(BDC)(H₂O)₂ and Zn(BDC)(H₂O) are present, as well as an unknown phase termed unknown structure 1 with a peak at 2θ=11° and 19.8°. The structure of unknown 1 could not be solved using single-crystal XRD. However, a structural formula of Zn(BDC) was proposed based on the TGA analysis. At 48 hours, Zn(BDC)(H₂O)₂ disappears and the proportion of unknown structure 1 (Zn(BDC)) in the mixture grows significantly, based on visual comparison. At 72 h, unknown structure 1 (Zn(BDC)) is the only phase identified, aside from the unreacted linker. The presence of the unknown structure 1 (Zn(BDC)) at long reaction times leads us to believe that this is the thermodynamically stable structure. The PXRDs at all reaction times show characteristic peaks of BDC at 2θ=17.3°, 25.2°, and 27.9°, in varying degrees, indicating that a molar ratio of 1:1 Zn:BDC is not enough to coordinate all of the generated BDC.

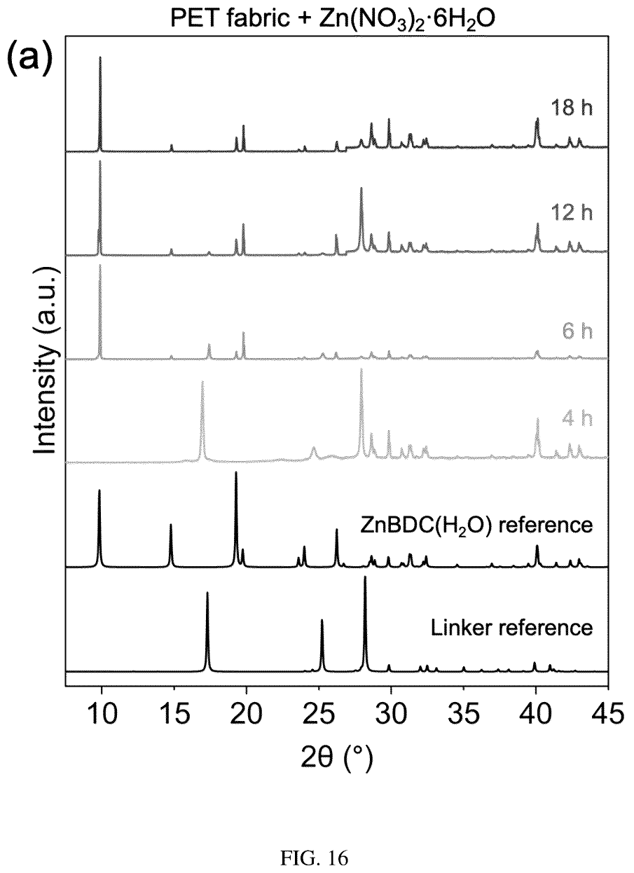

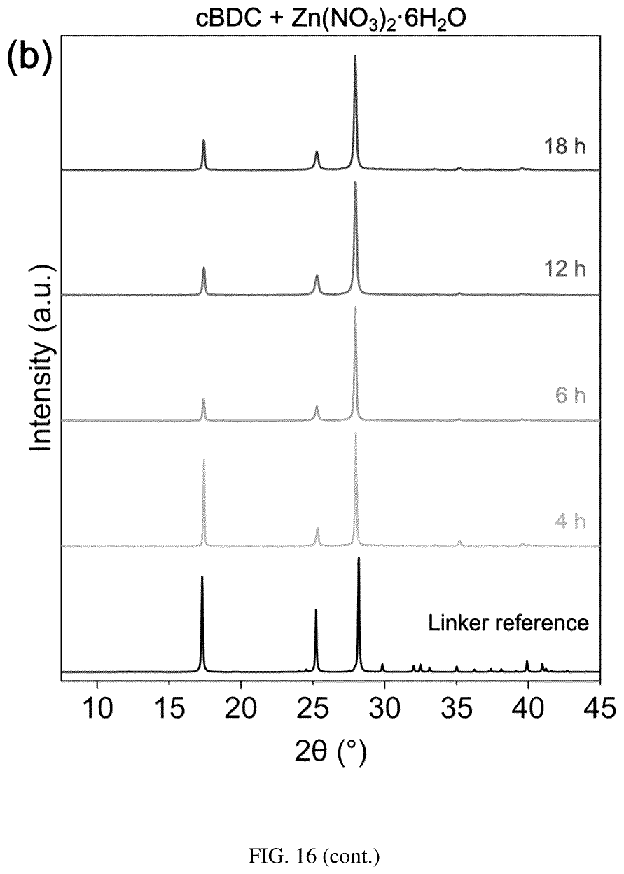

Recognizing that a 1:1 Zn:BDC ratio was insufficient to coordinate all the BDC, the molar ratio of Zn to BDC was doubled (to 2:1). At the shortest reaction time of 4 hours, there were only PXRD peaks for BDC and PET present (FIG. 16(a)). At 6 hours, MOF formation was initially observed; however, both PXRD and SEM images showed that BDC and PET were still the dominant solids. The first MOF formed at 6 hours, Zn(BDC)(H₂O) was not a stable phase as the reaction time was extended. At 24 hours and using a 2:1 Zn:BDC ratio, multiple structures are present, including unknown structure 1 (Zn(BDC)), Zn(BDC)(H₂O)₂, and a new phase, Zn₂(OH)₂(BDC)(H₂O), with the most intense peaks at 2θ=8.8° and 17.6°. At 48 and 72 hours, there is further transition to this new structure, Zn₂(OH)₂(BDC)(H₂O). This structure, along with Zn(BDC)(H₂O), persisted at 72 hours of reaction time. When doubling the Zn:BDC ratio again to 4:1, a complete conversion of the Zn-MOF to Zn₂(OH)₂(BDC)(H₂O) was observed after 6 hours, suggesting that the subsequent conversion from Zn(BDC)(H₂O) to Zn₂(OH)₂(BDC)(H₂O) is facilitated by excess Zn²⁺ in solution, which limited its formation at lower ratios of Zn:BDC. How the reaction stoichiometries and times affect the crystal phase was graphically represented in a phase diagram in FIG. 15(a).

As the reaction time increases, the intensities of BDC reflections decrease compared to the MOF peaks. This observation suggests that the Zn-MOF formation occurs only after PET hydrolysis begins and a critical BDC concentration is reached. To start, the Zn²⁺ ions likely catalyze the hydrolysis of PET fibers by weakening the PET-water interface and increasing the rate of hydrolysis. Additionally, the Lewis acidity of Zn²⁺ may contribute to the catalytic activity in the hydrolysis reaction. Zn²⁺, a Lewis acid, can coordinate with the carbonyl oxygen of the ester group in the PET polymer backbone. This coordination polarizes the carbonyl bond, making the carbonyl carbon more electrophilic and susceptible to nucleophilic attack by a water molecule. The Brønsted acidity of the hydrated zinc complex adds to the catalytic activity by protonating the ester oxygen of the PET backbone. This protonation further increases the electrophilicity of the carbonyl carbon, facilitating the nucleophilic attack by a water molecule. Together, these two acidities enhance the overall catalytic efficiency of Zn²⁺ in the hydrolysis of PET. This depolymerization step deposits BDC on the PET surface (the modified shrinking core model). The SEM images taken at 6 hours showed the fibers exhibited rough surfaces in contrast to smooth pristine PET fibers, likely due to BDC deposition. The BDC then coordinates the Zn²⁺ ions to form Zn-MOFs, freeing the surface for additional PET hydrolysis. The cycle continues until the PET hydrolysis is completed.

To gain better mechanistic insight into MOF formation, pristine, commercial BDC was used to synthesize Zn-MOFs in place of the PET fabric. Using identical conditions, a mixture of commercial BDC, Zn(NO₃)₂·6H₂O, and water (2:1:320) was heated at 210° C. at various times. Under these conditions, no formation of Zn-MOF was found at all the tested reaction times (FIG. 16(b)). When adding ethylene glycol, however, Zn-MOF formation was observed by PXRD. It was thus concluded that the EG released from PET hydrolysis plays a crucial role in MOF formation. Two hypotheses to explain this were identified. First, it is well known that EG can dissolve large amounts of multivalent-ion salts, including Zn²⁺, effectively increasing the Zn²⁺ concentration in solution. In addition, BDC has a higher solubility in EG than water, increasing the BDC solubility in the reaction mixture. Although Zn²⁺ is not saturated in the reaction mixture and is easily soluble in water, it is believed the EG is providing a mutual solvent for both Zn²⁺ and BDC, allowing for the reaction to proceed. Alternatively, at early reaction times, Zn²⁺ and EG might complex to form an insoluble coordination polymer of zinc glycolate (Zn(OCH₂CH₂O)). This polymer could provide a nucleation site or intermediate for Zn-MOF formation. Combining both observations, it was hypothesized that the Zn-MOF synthesis proceeds via an EG-assisted solvation of Zn ions and soluble BDC in the liquid phase.

Putting these observations together, the following formation mechanism of Zn-BDC MOFs from PET and Zn(NO₃)₂·6H₂O was proposed. The first step is the depolymerization of PET into BDC and EG, catalyzed by Zn²⁺. Detection of BDC in PXRD in several samples indicates that the BDC crystallizes and precipitates from the reaction mixture. Then, the presence of EG increases the solubility of BDC and Zn²⁺, allowing for the coordination of BDC to Zn ions in the solution. As the Zn-MOFs form, the solution runs out of BDC and Zn, causing additional BDC and Zn to dissolve. The observed initial Zn-BDC MOF phase, Zn(BDC)(H₂O), likely results from the first kinetic product crystallized out of solution under these conditions.

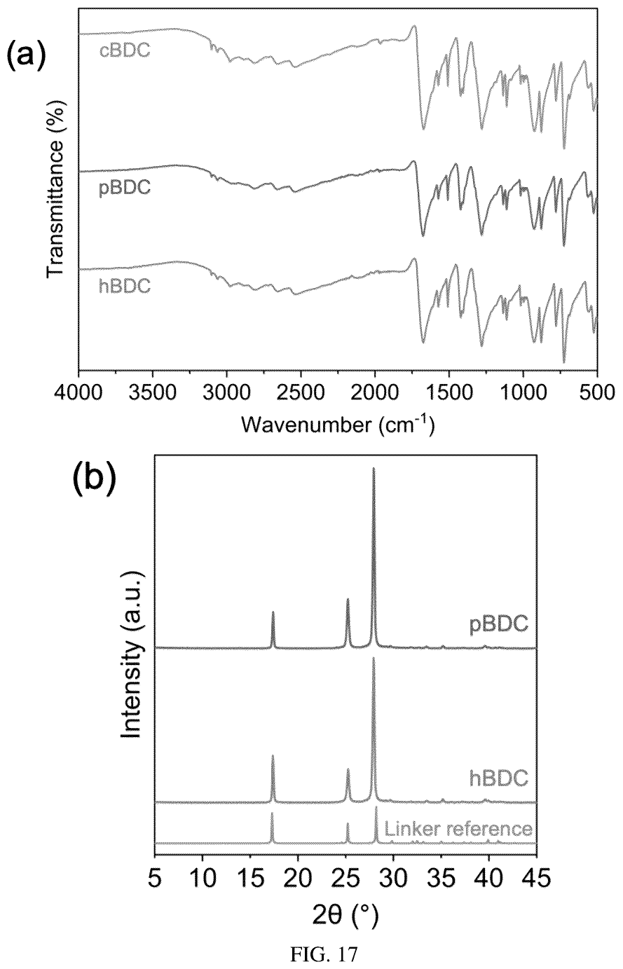

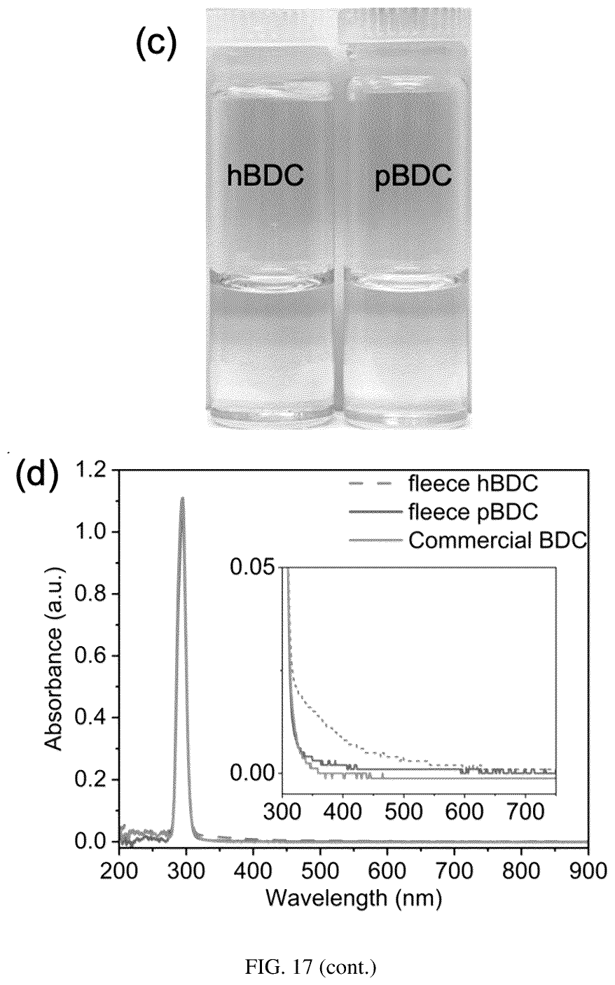

The experiments above were conducted with pre-consumer polyester twill; whether Zn-MOFs assembled from post-consumer PET fabric can purify BDC was investigated. Using a post-consumer fleece made of PET (black color), the PET textile, Zn(NO₃)₂·6H₂O, and water were mixed in a 1:1:320 molar ratio and heated at 210° C. for 12 h. PXRD of the prepared Zn-MOF matches well the Zn(BDC)(H₂O) features reported in the literature mixed with unreacted BDC. The yield of Zn(BDC)(H₂O) is 65% with respect to both Zn²⁺ and PET. As discussed above, Zn-MOF formation is limited by the amount of free Zn²⁺. The presence of unreacted BDC was, therefore, expected given the 1:1 Zn:BDC molar ratio. A goal was not necessarily to convert all PET to Zn-MOF but to demonstrate the recovery of clean BDC using minimal input chemicals (FIG. 17). The MOF/BDC suspension was centrifuged to separate the solid, pale-yellow MOF and BDC from the bright yellow supernatant solution. The supernatant solution likely consists largely of dyes and other additives. SEM showed that the obtained particles were on the order of 1-10 m with rectangular crystallite features. The MOF/BDC solid was subsequently rinsed with water to wash off any remaining remnants of impurities and with acetone to assist in drying.

Having demonstrated that a mixture of Zn-MOF and BDC can be obtained directly from post-consumer plastics, the Zn-MOF was disassembled by heating it in nitric acid. This process yields purified BDC (pBDC), which precipitated as a solid from an aqueous solution of Zn(NO₃)₂. Complete Zn-MOF to BDC conversion was found to occur at an equivalent 30:1 molar ratio of HNO₃:BDC at 100° C. for 24 h with stirring. The yield of pBDC was 93%±2.84. The PXRD of the disassembled product shows characteristic BDC peaks at 2θ=17.21, 25.01, and 27.64° (FIG. 17(b)). ¹H and ¹³C NMR also confirmed the presence of BDC. The purity of the pBDC sample dissolved in DMSO was assessed using UV-vis spectroscopy by comparing it against commercial BDC and BDC produced directly from hydrothermal hydrolysis of PET fleece fabric at 210° C. for 12 h (hBDC) (FIG. 17(d)). Comparing the absorbance spectra of pBDC to commercial BDC, both have the characteristic UV-absorbance features of BDC with no other absorbance features. hBDC, on the other hand, exhibits additional absorbance between 350-500 nm, indicating the presence of impurities such as dyes. These impurities can also be seen as a yellow hue in the solution in the photo in see FIG. 17(c)). pBDC was further tested for Zn impurities using X-ray photoelectron spectroscopy (XPS). This test showed Zn impurities of less than 0.01 atomic percent (referenced against carbon and oxygen. This observation suggests that pBDC has a purity close to the commercial BDC monomers, validating the hypothesis that a cycle of Zn-MOF assembly-disassembly could be used to turn PET into impurity-free BDC monomers.

A one-step depolymerization-crystallization of post-consumer PET textiles using a Zn-BDC MOF as an intermediate was developed. The recovery of purified BDC, a PET monomer, following the acid-induced Zn-MOF disassembly, without needing base addition, was demonstrated. The formation of Zn-BDC MOFs greatly depended upon reaction conditions. This sensitivity was attributed to the MOF assembly mechanism, which for Zn-BDC MOF, requires BDC to dissolve and coordinate with Zn²⁺ ions. Owing to the need for the solution-phase complexation pathway, adjusting the Zn:BDC molar ratio and reaction time significantly affected the final Zn-BDC MOF crystal structures. It was also found that PET cannot react directly with Zn ions to form Zn-BDC MOF. Instead, the PET must first undergo hydrolysis to create BDC. Only when BDC is present in solution can the MOF begin to form. The transformation cycle thus cascades through PET hydrolysis, BDC deposition-precipitation, BDC dissolution, Zn-BDC complexation, and Zn-MOF formation. In the case of PET, the presence of byproduct ethylene glycol, the other monomer of PET, likely helps promote the synthesis by increasing the solubility of Zn²⁺ and BDC in the solvent.

Although the present disclosure has been described with respect to one or more particular embodiments and/or examples, it will be understood that other embodiments and/or examples of the present disclosure may be made without departing from the scope of the present disclosure.

1. A method of forming one or more purified monomer(s) and/or one or more structural analog(s) thereof comprising:
forming one or more metal complex(es), one or more metal organic framework(s), one or more coordination polymer network(s), or any combination thereof using one or more impure monomer(s), structural analog(s) thereof, or any combination thereof;
optionally, isolating the metal complex(es), the metal organic framework(s), the coordination polymer network(s), or the combination thereof;
optionally, decomposing the metal complex(es), the metal organic framework(s), coordination polymer network(s), or the combination thereof,
wherein the purified monomer(s) and/or the structural analog(s) thereof are formed; and
optionally, isolating the purified monomer(s) and/or the structural analog(s) thereof.

2. The method of claim 1, wherein the forming the metal complex(es) comprises forming one or more first metal complex(es), one or more first metal organic framework(s), one or more first coordination polymer network(s), or any combination thereof using one or more first impure monomer(s), structural analog(s) thereof, or any combination thereof and then forming one or more second metal complex(es), one or more second metal organic framework(s), one or more second coordination polymer network(s), or any combination thereof is carried out using one or more second impure monomer(s), structural analog(s) thereof, or any combination thereof is carried out.

3. The method of claim 1, the method further comprising providing the impure monomer(s), the structural analog(s) thereof, or any combination thereof prior to the forming the metal complex(es), the metal organic framework(s), the coordination polymer network(s), or the combination thereof.

4. The method of claim 3, wherein the providing the impure monomer(s), the structural analog(s) thereof, or the combination thereof comprises depolymerizing one or more polymer(s).

5. The method of claim 4, wherein the providing the impure monomer(s), the structural analog(s) thereof, or the combination thereof and the forming the metal complex(es), the metal organic framework(s), the coordination polymer network(s), or the combination thereof are carried out in a single reaction mixture and/or without isolation of the impure monomer(s), the structural analog(s) thereof, or the combination thereof.

6. The method of claim 4, wherein the polymer(s) is/are independently chosen from organic polymers, biopolymers, and any combination thereof.

7. The method of claim 1, wherein the impure monomer(s), the structural analog(s) thereof, or the combination thereof is/are organic acid(s), polyol(s), amine(s), or any combination thereof.

8. The method of claim 1, wherein the impure monomer(s), the structural analog(s) thereof, or the combination thereof is/are independently formed from a textile, an article of manufacture, or any combination thereof.

9. The method of claim 1, wherein the forming the metal complex(es), the metal organic framework(s), the coordination polymer network(s), or the combination thereof comprises contacting the impure monomer(s), the structural analog(s) thereof, or the combination thereof, with one or more metal precursor(s), wherein the metal complex(es), the metal organic framework(s), the coordination polymer network(s), or the combination thereof are formed.

10. The method of claim 1, wherein the decomposing the metal complex(es), the metal organic framework(s), the coordination polymer network(s), or the combination thereof comprises contacting the metal complex(es), the metal organic framework(s), the coordination polymer network(s), or the combination thereof with one or more acid(s), one or more base(s), one or more organic solvent(s), or one or more ionic liquid(s), or any combination thereof.

11. The method of claim 1, wherein the decomposing the metal complex(es), the metal organic framework(s), the coordination polymer network(s), or the combination thereof comprises contacting the metal complex(es), the metal organic framework(s), the coordination polymer network(s), or the combination thereof with one or more acid(s), wherein a metal precursor or precursors or a structural analog or analogs thereof are formed.

12. The method of claim 1, wherein the impure monomer(s), the structural analog(s) thereof, or the combination thereof comprise(s) one or more impurit(ies) and the purified monomer(s) and/or structural analog(s) thereof is/are substantially free or free of the impurit(ies) and/or the impure monomer(s), the structural analog(s) thereof, or the combination thereof comprises(es) one or more additive(s) and the purified monomer(s) and/or structural analog(s) thereof is/are substantially free or free of the additive(s).

13. The method of claim 1, wherein the impure monomer(s), the structural analog(s) thereof, or the combination thereof are less than 99% pure by weight.

14. The method of claim 10, wherein the impure monomer(s), the structural analog(s) thereof, or the combination thereof are less than 90% pure by weight.

15. The method of claim 1, wherein the purified monomer(s) and/or structural analog(s) thereof is/are substantially colorless or colorless.

16. The method of claim 1, further comprising polymerizing the purified monomer(s) and/or structural analog(s) thereof.

17. The method of claim 5, wherein the single reaction mixture comprises one or more metal salt(s), one or more polymer-containing material(s), wherein each of the polymer-containing material(s) independently comprises the one or more polymer(s) and one or more impurities, and optionally an aqueous solvent or steam, and wherein the polymer-containing material(s) is/are chosen from textiles, articles of manufacture, and any combination thereof.

18. The method of claim 1, wherein the forming the one or more metal complex(es), the one or more metal organic framework(s), the one or more coordination polymer network(s), or the combination thereof using one or more impure monomer(s), structural analog(s) thereof, or any combination thereof comprises:
mixing one or more metal salt(s), one or more polymer-containing material(s), wherein each of the polymer-containing material(s) independently comprises the one or more polymer(s), and one or more impurities, and optionally an aqueous solvent to form a mixture; and
reacting the mixture in thermal degradation condition(s) to form a thermal degraded or a hydrolyzed mixture, such that the polymer(s) in the polymer-containing material(s) depolymerize(s) to release one or more monomer(s) mixed with one or more impurities forming one or more impure monomer(s), wherein the one or more monomer(s) react with the metal salt(s) to form the one or more metal complex(es), the one or more metal organic framework(s), the one or more coordination polymer network(s), or the combination thereof, and the one or more impurit(ies) is/are substantially excluded from the one or more metal complex(es), the one or more metal organic framework(s), the one or more coordination polymer network(s), or the combination thereof.

19. The method of claim 18, wherein the thermal degradation condition(s) comprise(s) a temperature of about 100° C. to about 300° C. and optionally a water- or steam-containing environment.

20. The method of claim 18, wherein a reaction time of reacting the mixture in the thermal degradation condition(s) is about 1 hour to about 3 days.

21. The method of claim 18, wherein the depolymerizing the polymer(s) to release the one or more monomer(s) and/or the reacting the released one or more monomer(s) with the metal salt(s) is/are in one pot, such that the one or more monomer(s) is/are substantially converted to the one or more metal complex(es), the one or more metal organic framework(s), the one or more coordination polymer network(s), or the combination thereof without keeping accumulated in the thermal degraded or the hydrolyzed mixture comprising the metal salt(s).

22. The method of claim 18, further comprising:
washing the thermal degraded or the hydrolyzed mixture or one or more crystalline material(s) independently comprising the one or more metal complex(es), the one or more metal organic framework(s), the one or more coordination polymer network(s), or the combination thereof in the thermal degraded or the hydrolyzed mixture with one or more solvent(s) to remove the one or more impurit(ies) and obtain a purified metal complex(es), metal organic framework(s), coordination polymer network(s), or any combination thereof; and
decomposing the purified metal complex(es), the metal organic framework(s), coordination polymer network(s), or the combination thereof to produce a purified monomer(s) and/or structural analog(s) thereof substantially free of the one or more impurities.

23. A polymer formed from at least a purified monomer or purified monomers formed by a method of claim 1.

24. The polymer of claim 23, wherein the polymer is chosen from organic polymers and biopolymers.

25. The polymer of claim 24, wherein the organic polymer comprises a polyester, a polylactic acid, a polyamide, a polyalcohol, a polyurethane, a polyurea, a polycarbonate, a polyether ether ketone, a polyether ether ketone ketone, a polyether ketone ketone, a polyetherimide, a polystyrene, or any combination thereof.

26. The polymer of claim 23, wherein the polymer is substantially free of impurit(ies) and/or additive(s) present in the impure monomer(s) from which the purified monomer(s) was/were formed.

27. An article of manufacture comprising one or more polymer(s) of claim 23.

28. The article of manufacture of claim 27, wherein the article of manufacture is a container, a structural element, or a surface element.

29. The article of manufacture of claim 28, wherein the container is a bottle, a food tray, or a food packaging.

30. The article of manufacture of claim 28, wherein the surface element is a component of or part from an automobile or a plane.
